SEQUENCE LISTING (67 sequences)

SEQ ID NO: 1 - DNA, 1497 bp, Alstroemeria
atggttctctccagcgccgtctccgacaacaccggcccagtccactgcaccttcgcgtcgcgctacgtccgcgatgcgcctgcccggttcaggatgccggagaattcgatacccaaggacacggcgtaccagatcgtcaacgacgagctgatgctggacgggaaccccaggctgaacctggcgtcgttcgtcacgacctggatggagccggagtgcgatcgcctcatgatcgccgccgccaacaagaactatgtcgacatggacgagtacccggtcacaaccgagctacagaatcgctgtgttaatataatagcccacctcttcaatgcgcctattggggatgaggaaacagcagtaggagttgggacagtggggtcatcagaagcaataatgcttgcaggcttggcattcaagagaaagtggcagaacaaaaaaaaagcagaggggaagccatatgacaagcccaatattgtcaccggtgcaaacgttcaggtttgctgggagaaattcgctaggtattttgaagttgaactgaaagaagtgaagctgagggagggttattacatcatggacccagagaaggctgtggaaatggtggatgagaataccatatgtgttgctgctatcttgggctcaacccttactggagagttcgaagatgttaaactactgaacaaacttcttgaagagaagaacaaggaaactgggtgggacacacccattcatgttgatgctgctagtggtggattcattgctccttttctatacccagaactggaatgggatttccgattaccactggtgaagagtattaatgtcagcggacacaaatatggccttgtttatgcaggtgtgggttgggttgtctggaggaacaaagaagatcttcctgaagagctcattttccatataaactaccttggggcagatcagcctactttcaccctcaatttctctaaaggttcaagccagataattgctcaatattatcaattcattcgccttggttttcaggggtataagaacataatggaaaactgcatggagaacacaagaatactgagagaaggtctgcaggagacgggccgtttcgagatagtctccaaagatattggggtgcctcttgttgcatttgctctcaaggacagcagccagtacactgtctttgagatagcggacgccatgagaaggttcggatggatcattcctgcatacaccatgccaaaggacgcggagcacatagctgtcctccgtgtggttatcagggaggatttcagcaggagccttgctgagcgcctagttaatgacatgaagaaggtgctggctgagctggacgtacttcccagtcgcatcaccaccattgcccatgttacggctgtggagaacgataatggcgaagctgtgatcaagaagagtttcctggagatagagaagaaggttattacacattggaaggatgtagtgatgaacggcaagaagactaataaagtttgctga

SEQ ID NO: 2 - protein, 498 aa, Alstroemeria
MVLSSAVSDNTGPVHCTFASRYVRDAPARFRMPENSIPKDTAYQIVNDELMLDGNPRLNLASFVTTWMEPECDRLMIAAANKNYVDMDEYPVTTELQNRCVNIIAHLFNAPIGDEETAVGVGTVGSSEAIMLAGLAFKRKWQNKKKAEGKPYDKPNIVTGANVQVCWEKFARYFEVELKEVKLREGYYIMDPEKAVEMVDENTICVAAILGSTLTGEFEDVKLLNKLLEEKNKETGWDTPIHVDAASGGFIAPFLYPELEWDFRLPLVKSINVSGHKYGLVYAGVGWVVWRNKEDLPEELIFHINYLGADQPTFTLNFSKGSSQIIAQYYQFIRLGFQGYKNIMENCMENTRILREGLQETGRFEIVSKDIGVPLVAFALKDSSQYTVFEIADAMRRFGWIIPAYTMPKDAEHIAVLRVVIREDFSRSLAERLVNDMKKVLAELDVLPSRITTIAHVTAVENDNGEAVIKKSFLEIEKKVITHWKDVVMNGKKTNKVC

SEQ ID NO: 3 - DNA, 1530 bp, Alstroemeria
atggctctctccagcgtcgtctccgactccaacaaccaagtgcagtgcacctatgcctctcgctacgttcgcgacgaggctccagggttcaggatgccggagaagtcgataccaaaggaggcggcgttcacgatgatcaacgacgagctgatgctggacgggaaccccaggctgaacttggcttcgttcgtgacgacgtggatggagccggagtgcgatcgtctgatgatgtccaccatcaacaagaactacgccctcatggacgattacccggtcactattgacatacagaatcgctgcgtgaatatgatagccaacctctttaatgcgccaattggggagggggaaacaacagtaggatgtgctacggtgggatcatcagaagccatgatgcttgcagggttggcattcaagagaaattggcagaacaaaagaaaggcagaggggaagccatatgacaagcccaacatggtcaccggttcaaatgttcaggtttgctgggtgaaattcgctaagtattttgaagttgaaatgaaaaaagtgaatttgagggagggatattatgtgatggacccagagaaggctgtggaaatggtggatgagaataccatttgtgttgctgccatcttgggctcgacccttactggagagttcgaagatgtcaaactgctaaacgacctcctcgtagagaagaacaagaaaactggttgggatacacccattcatgttgatgctgctattggtggattcattgctccatttatctatccagaactggaatgggatttccgactacctctggtgaagagtatcaatgtcagtggacacaaatacggccttgtctatcccggcgttggttgggtcgtctggaggaacaagaatgatcttcctgaagaactcattttccatatcaactatcttgggattgatcaacccacttttaccctcaacttctcgaaaggttcaaaccagataattggtcaatactatcaattaattcgccttggttttgaggggtacaagaatataatggaaaactgcacggagaatgcaagaattcttagggaacatctcgaggagatgggcgttttcgagatcatctccaaggatattggggcgcctctcgtcacaattgctctcaaggacagcagcaaacatagtgtctttaagatagccgatacaattagaaggtttggatggacaattcctgcatacacaatgccaaaggacgttgagcacatagccgtccttcgtgtggttatcagggaggacttcagccggagcctcgccgagcgcctagctaatgacatgaagaaggtgttggttgagctggacatacatcccagtcgcaccaccaccattgcccacgttaaggcagtggagaatggcaatggtaattatgtgatcaagaagagtattgtagagaatggcaatggcgaacatgtgatcaagaaaagcattggggagaatggcaatggcaaacacgcgatcaagaattgcagggatgcagtgattgcaaacgcggcatga

SEQ ID NO: 4 - protein, 509 aa, Alstroemeria
MALSSVVSDSNNQVQCTYASRYVRDEAPGFRMPEKSIPKEAAFTMINDELMLDGNPRLNLASFVTTWMEPECDRLMMSTINKNYALMDDYPVTIDIQNRCVNMIANLFNAPIGEGETTVGCATVGSSEAMMLAGLAFKRNWQNKRKAEGKPYDKPNMVTGSNVQVCWVKFAKYFEVEMKKVNLREGYYVMDPEKAVEMVDENTICVAAILGSTLTGEFEDVKLLNDLLVEKNKKTGWDTPIHVDAAIGGFIAPFIYPELEWDFRLPLVKSINVSGHKYGLVYPGVGWVVWRNKNDLPEELIFHINYLGIDQPTFTLNFSKGSNQIIGQYYQLIRLGFEGYKNIMENCTENARILREHLEEMGVFEIISKDIGAPLVTIALKDSSKHSVFKIADTIRRFGWTIPAYTMPKDVEHIAVLRVVIREDFSRSLAERLANDMKKVLVELDIHPSRTTTIAHVKAVENGNGNYVIKKSIVENGNGEHVIKKSIGENGNGKHAIKNCRDAVIANAA

SEQ ID NO: 5 - DNA, 1590 bp, Alstroemeria
atggctctctccagcgtcgtctccgactccaacaaccaagtgcagtgcacctatgcctctcgctacgttcgcgacgaggctccagggttcaggatgccggagaagtcgataccaaaggaggcggcgttcacgatgatcaacgacgagctgatgctggacgggaaccccaggctgaacttggcttcgttcgtgacgacgtggatggagccggagtgcgatcgtctgatgatgtccaccatcaacaagaactacgccctcatggacgattacccggtcactattgacatacagaatcgctgcgtgaatatgatagccaacctctttaatgcgccaattggggagggggaaacaacagtaggatgtgctacggtgggatcatcagaagccatgatgcttgcagggttggcattcaagagaaattggcagaacaaaagaaaggcagaggggaagccatatgacaagcccaacatggtcaccggttcaaatgttcaggtttgctgggtgaaattcgctaagtattttgaagttgaaatgaaaaaagtgaatttgagggagggatattatgtgatggacccagagaaggctgtggaaatggtggatgagaataccatttgtgttgctgccatcttgggctcgacccttactggagagttcgaagatgtcaaactgctaaacgacctcctcgtagagaagaacaagaaaactggttgggatacacccattcatgttgatgctgctattggtggattcattgctccatttatctatccagaactggaatgggatttccgactacctctggtgaagagtatcaatgtcagtggacacaaatacggccttgtctatcccggcgttggttgggtcgtctggaggaacaagaatgatcttcctgaagaactcattttccatatcaactatcttgggattgatcaacccacttttaccctcaacttctcgaaaggttcaaaccagataattggtcaatactatcaattaattcgccttggttttgaggggtacaagaatataatggaaaactgcacggagaatgcaagaattcttagggaacatctcgaggagatgggcgttttcgagatcatctccaaggatattggggcgcctctcgtcacaattgctctcaaggacagcagcaaacatagtgtctttaagatagccgatacaattagaaggtttggatggacaattcctgcatacacaatgccaaaggacgttgagcacatagccgtccttcgtgtggttatcagggaggacttcagccggagcctcgccgagcgcctagctaatgacatgaagaaggtgttggttgagctggacatacatcccagtcgcaccaccaccattgcccacgttaaggcagtggagaatggcaatggtaattatgtgatcaagaagagtattgtagagaatggcaatggcgaacatgtgatcaagaaaagcattggggagaatggcaatggcaaacacgcgatcaagaatgaacatgtgatcaagaagagcattggggagaatttcaatggcaaacatgcaatcaagaattgcagggatgcagtgattgcaaacgcggcatga

SEQ ID NO: 6 - protein, 529 aa, Alstroemeria
MALSSVVSDSNNQVQCTYASRYVRDEAPGFRMPEKSIPKEAAFTMINDELMLDGNPRLNLASFVTTWMEPECDRLMMSTINKNYALMDDYPVTIDIQNRCVNMIANLFNAPIGEGETTVGCATVGSSEAMMLAGLAFKRNWQNKRKAEGKPYDKPNMVTGSNVQVCWVKFAKYFEVEMKKVNLREGYYVMDPEKAVEMVDENTICVAAILGSTLTGEFEDVKLLNDLLVEKNKKTGWDTPIHVDAAIGGFIAPFIYPELEWDFRLPLVKSINVSGHKYGLVYPGVGWVVWRNKNDLPEELIFHINYLGIDQPTFTLNFSKGSNQIIGQYYQLIRLGFEGYKNIMENCTENARILREHLEEMGVFEIISKDIGAPLVTIALKDSSKHSVFKIADTIRRFGWTIPAYTMPKDVEHIAVLRVVIREDFSRSLAERLANDMKKVLVELDIHPSRTTTIAHVKAVENGNGNYVIKKSIVENGNGEHVIKKSIGENGNGKHAIKNEHVIKKSIGENFNGKHAIKNCRDAVIANAA

SEQ ID NO: 7 - DNA, 1416 bp, tulip pistil
atgatgccgctggcctgctccgatcccagcccgccacccaggttcagagggcatgctatgttggcgccgttcacggctgggtggcagacagccgatacggaacccttcatcattacaagatctgagggttgttacgtttatgacattaccggcaagaaataccttgactctcttgctggattgtggtgcacggctttaggtggaagtgaacctcgcctcattgcagctgcaactgaacaactaaaccagttgccattttaccactcgttttggaatcgtaccacaaagccatctttggatcttgcaacggaacttattgcatatttcactccaaagaaaatggggaaagtcttctttacaaatagtggttcagaggctaatgattctcaggtgaagctcgtttggtattataataacgctttgggaagaccaaataagaagaaattcatagcaagaacaaaatcgtaccacggggtgacattgacatcagctagtctcactggtcttccggctctacatcaaaagttcgatcttccattaccatttgtgttgcacacagattgtccacactattggcgcttccatctaccgggtgagacggaggaggagttttcgacgaggctagctaataacttggagaaactaatcctcacagagggaccagaaacaattgctgcatttatcgcagagcctgtcatgggtgctggaggtgttatccctcctcccaaaacctattttgagaagattcaagccgtcataaagaaatatgacattctcttcatcgctgatgaggtcgttactgcctttggtaggctagggacaatgtttggatgtgaaaaatacaatattcagcctgaccttgtaaccatagcaaaagctctttcttctggatacctacctattggtgcaatacttgtgagtcctgaaatagcagaagtcgtacattctcaaagcaacaaactcggttccttttctcatggatttacatattcgggacatccagtagcctgtgctgttgctttagaagcactaaaaatatacaaggaaagggatattccgggccatgtccaaaccatatctcccagattccaagatggtctcagagccttctctgatagctcgataattggcgagatacgtggaacaggattaattctggcgactgagttcactgacaacaagtctcctaatgacctattcccatctgagtggggagtaggtgcaatattcggagcagagtgtgcaaagcgtggactgctggttcgagttgctggggataacataatgatgtcacctccgcttgtaatatccccgaaagaagttgatgagctagtaagcatttacggggaagcccttaaatgtacagaagagagggtagctgagctcaaagactcagaaatagcagtagcaaagtga

SEQ ID NO: 8 - protein, 471 aa, tulip pistil
MMPLACSDPSPPPRFRGHAMLAPFTAGWQTADTEPFIITRSEGCYVYDITGKKYLDSLAGLWCTALGGSEPRLIAAATEQLNQLPFYHSFWNRTTKPSLDLATELIAYFTPKKMGKVFFTNSGSEANDSQVKLVWYYNNALGRPNKKKFIARTKSYHGVTLTSASLTGLPALHQKFDLPLPFVLHTDCPHYWRFHLPGETEEEFSTRLANNLEKLILTEGPETIAAFIAEPVMGAGGVIPPPKTYFEKIQAVIKKYDILFIADEVVTAFGRLGTMFGCEKYNIQPDLVTIAKALSSGYLPIGAILVSPEIAEVVHSQSNKLGSFSHGFTYSGHPVACAVALEALKIYKERDIPGHVQTISPRFQDGLRAFSDSSIIGEIRGTGLILATEFTDNKSPNDLFPSEWGVGAIFGAECAKRGLLVRVAGDNIMMSPPLVISPKEVDELVSIYGEALKCTEERVAELKDSEIAVAK

SEQ ID NO: 9 - DNA, 1524 bp, Alstroemeria
atgatgatcgccggcaagctcctccgatcaaaggcctccggccaggctggagctctcgtgaggaacgcgctgcgcgggctctcgccttgctccgcggtcgcgggagcgatcgccgcgccgtcggcgagagtgttcggctcagcggcggagctgagagatgagagagggtacaaggggcatggcatgctggcgccgttcacggccggatggcagagcaacgacgtccaccctctgatcatcgagagatccgagggtgtctatgtttatgacaacaatggaaataagtatcttgattctctcgctggattatggtgcactgctctaggaggcaatgagcctcgccttgttgcagctgcaactgctcagttaaataaactgccattttatcactccttttggaatcgtactacgataccctccttggatcttgcaaaggagattctagagtttttcacggtaaagaagatgggcaaggtcttcttcacaaacagtggctcagaagcaaatgattcccaggtgaaattggtttggtattataacaacgcactgggaagaccaaataagaaaaaattcatagcaagatcaaaatcataccacgggtcgacgttgataactgctagtcttactggccttcctgcactacatcaaaagtttgatctgccagcaccatttgttttacacacagattgtccgcattattggcgataccatttaccaggcgagacagaggagaaattttcaaccaggttggccaataacttggagaaccttatcgtcaaagagggaccagatacgattgccgcatttatcgctgagcctgtcatgggtgctggaggtgttatacctcccccaaaatcgtattttgaaaaggtccaagcaatcgtgaagaagtatgatatcctcttcattgcagacgaggtcgttactgcttttggaaggttggggacaatgttcggatgtgaaaagtacaatattcagcctgatcttgtctcgatcgcaaaagctctttcatcggcatacatgccaattggtgccattcttgtcagttcagaaatatctgatgtaataaactctcaaagtaacaaacttggtatattcgctcatggttttacatattctgggcatccggtatcctgtgctgttgctctagaagcactaaaaatatacaaggaaagaaatatacctgagcatgtccggtctgtgtctccaagatttcaagatggcctccgagcgttctcagacagtcccataattggcgagatacgcgggactggcatgattcttgcgactgagttcacggagaacaagtctccagatcatcctttccctcccgaatggggcgtcggagcaatatttggggcagaatgccagaaacgcggattactggttcgagttgctggagatgcgataatgatgtcacctccattgacaataactcctaatgaaattgacgagctaataagcatatacggcgaagccttgaagcaaacggagaagagggtgaaggagttaaaatctcagtaa

SEQ ID NO: 10 - protein, 507 aa, Alstroemeria
MMIAGKLLRSKASGQAGALVRNALRGLSPCSAVAGAIAAPSARVFGSAAELRDERGYKGHGMLAPFTAGWQSNDVHPLIIERSEGVYVYDNNGNKYLDSLAGLWCTALGGNEPRLVAAATAQLNKLPFYHSFWNRTTIPSLDLAKEILEFFTVKKMGKVFFTNSGSEANDSQVKLVWYYNNALGRPNKKKFIARSKSYHGSTLITASLTGLPALHQKFDLPAPFVLHTDCPHYWRYHLPGETEEKFSTRLANNLENLIVKEGPDTIAAFIAEPVMGAGGVIPPPKSYFEKVQAIVKKYDILFIADEVVTAFGRLGTMFGCEKYNIQPDLVSIAKALSSAYMPIGAILVSSEISDVINSQSNKLGIFAHGFTYSGHPVSCAVALEALKIYKERNIPEHVRSVSPRFQDGLRAFSDSPIIGEIRGTGMILATEFTENKSPDHPFPPEWGVGAIFGAECQKRGLLVRVAGDAIMMSPPLTITPNEIDELISIYGEALKQTEKRVKELKSQ

SEQ ID NO: 11 - DNA, 1281 bp, Escherichia coli
atgaacagcaataaagagttaatgcagcgccgcagtcaggcgattccccgtggcgttgggcaaattcacccgattttcgctgaccgcgcggaaaactgccgggtgtgggacgttgaaggccgtgagtatcttgatttcgcgggcgggattgcggtgctcaataccgggcacctgcatccgaaggtggtggccgcggtggaagcgcagttgaaaaaactgtcgcacacctgcttccaggtgctggcttacgagccgtatctggagctgtgcgagattatgaatcagaaggtgccgggcgatttcgccaagaaaacgctgctggttacgaccggttccgaagcggtggaaaacgcggtaaaaatcgcccgcgccgccaccaaacgtagcggcaccatcgcttttagcggcgcgtatcacgggcgcacgcattacacgctggcgctgaccggcaaggtgaatccgtactctgcgggcatggggctgatgccgggtcatgtttatcgcgcgctttatccttgcccgctgcacggcataagcgaggatgacgctatcgccagcatccaccggatcttcaaaaatgatgccgcgccggaagatatcgccgccatcgtgattgagccggttcagggcgaaggcggtttctacgcctcgtcgccagcctttatgcagcgtttacgcgctctgtgtgacgagcacgggatcatgctgattgccgatgaagtgcagagcggcgcggggcgtaccggcacgctgtttgcgatggagcagatgggcgttgcgccggatcttaccacctttgcgaaatcgatcgcgggcggcttcccgctggcgggcgtcaccgggcgcgcggaagtaatggatgccgtcgctccaggcggtctgggcggcacctatgcgggtaacccgattgcctgcgtggctgcgctggaagtgttgaaggtgtttgagcaggaaaatctgctgcaaaaagccaacgatctggggcagaagttgaaagacggattgctggcgatagccgaaaaacacccggagatcggcgacgtacgcgggctgggggcgatgatcgccattgagctgtttgaagacggcgatcacaacaagccggacgccaaactcaccgccgagatcgtggctcgcgcccgcgataaaggcctgattcttctctcctgcggcccgtattacaacgtgctgcgcatccttgtaccgctcaccattgaagacgctcagatccgtcagggtctggagatcatcagccagtgttttgatgaggcgaagcagtag

SEQ ID NO: 12 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 13 - DNA, 870 bp, tulip pistil
atggaggtgggattcctgggcctcggcatcatggggaaggcgatggccgtcaacctcctccgctccggcttccgcgtcaccgtctggaaccggaccctctccaagtgcaatgagctactggaacaaggtgcttctgttggagaaaccccagcagctgtaataaagaagtgcaaatataccatagcaatgctatctgatcctagtgccgctctttcggttgtttttgacaaagacggggtacttgagcatatgtctagcggaaaaggctatattgacatgtcaacagttgatgcagttacttcatccaagatcagcgaggctattacacagaagggtgggcatttccttgaagctcccgtatcaggtagcaaaaagccagctgaggatggacagctagttattcttgctgccggggagaaagcattgtatgaagaaataattcctgcatttgaagtattaggaaaaaaatctttctttttgggacaagtgggaaatggtgcaaacatgaagctcatagtaaacatgatcatgggcagtatgatgaatgcactatctgaaggactcagcttggctggcaaaagtggacttgagcagaaaacgcttcttgacgtgctggatcttggtgccattgctaacccaatgttcaagttaaaaggtcctgccatgatccaaaataatcaccctccagcattccccctcaaacatcaacagaaggatatgagattggctctcgctcttggcgacgagaatgctgtctcaatgccagttgctgctgctgccaatgaggcatttaagaaggctaggagcctggggctgggggaccttgatttctcggccgtatacgaagtattgaagtatggagatgcatcg

SEQ ID NO: 14 - protein, 290 aa, tulip pistil
MEVGFLGLGIMGKAMAVNLLRSGFRVTVWNRTLSKCNELLEQGASVGETPAAVIKKCKYTIAMLSDPSAALSVVFDKDGVLEHMSSGKGYIDMSTVDAVTSSKISEAITQKGGHFLEAPVSGSKKPAEDGQLVILAAGEKALYEEIIPAFEVLGKKSFFLGQVGNGANMKLIVNMIMGSMMNALSEGLSLAGKSGLEQKTLLDVLDLGAIANPMFKLKGPAMIQNNHPPAFPLKHQQKDMRLALALGDENAVSMPVAAAANEAFKKARSLGLGDLDFSAVYEVLKYGDAS

SEQ ID NO: 15 - DNA, 867 bp, Arabidopsis
atggaagtagggtttctgggtttgggaatcatgggaaaagccatgtcaatgaatctattgaagaatggattcaaagtcactgtatggaacagaacactctccaagtgtgatgagcttgtggagcatggtgcatcagtatgtgagagtccagctgaagtaatcaagaaatgcaaatacactattgctatgctctctgatccttgtgctgctctttcggttgttttcgataaaggcggtgttttggagcagatatgtgaaggaaaaggttatatcgatatgtcgactgttgatgcagagacttctttgaagatcaatgaggcaatcaccgggaagggtggtcggttcgtagaaggtccggtttcaggtagcaaaaagccagctgaagatggccaactcattatccttgctgctggtgacaaggcactctttgaggaatcaatcccagcttttgatgtcttggggaagagatcgttttacttgggacaagttggaaacggagctaaaatgaagctaatagtgaacatgataatgggaagcatgatgaatgcattctctgaggggcttgtattggctgacaagagtggacttagctctgacactcttttggatattctggatctgggagcaatgactaacccgatgttcaaggggaaaggaccttcaatgaacaagagtagttacccaccagcatttccattgaaacatcagcagaaagacatgaggctagctcttgctcttggcgatgaaaacgcggtttccatgcctgtagccgcggctgcaaacgaggcttttaagaaggcgagaagcttgggactaggagatctcgacttctctgctgtgattgaagctgtgaaattctcccgcgaa

SEQ ID NO: 16 - protein, 289 aa, Arabidopsis
MEVGFLGLGIMGKAMSMNLLKNGFKVTVWNRTLSKCDELVEHGASVCESPAEVIKKCKYTIAMLSDPCAALSVVFDKGGVLEQICEGKGYIDMSTVDAETSLKINEAITGKGGRFVEGPVSGSKKPAEDGQLIILAAGDKALFEESIPAFDVLGKRSFYLGQVGNGAKMKLIVNMIMGSMMNAFSEGLVLADKSGLSSDTLLDILDLGAMTNPMFKGKGPSMNKSSYPPAFPLKHQQKDMRLALALGDENAVSMPVAAAANEAFKKARSLGLGDLDFSAVIEAVKFSRE

SEQ ID NO: 17 - DNA, 1365 bp, tulip pistil
atggttgtccagggaagccatgtcctcatcgtcccctaccccggccaaggtcacctaaaccctatgctgcagttcggcaagcacctcgcacaccatggccttactgtcaccgtcgccaccacccgttacatcctctccactaaccctcccgaccccaaactcacccatattactttcgcccccatttccgacggcttcgacgatggaggctttggcgcctgcggggatgttacgtcctatctcgcaaggatggagtcggtcgggtcccagtccttgacggacctcatcgagtcaagcagtgcagagggccgccccgtccgggtaatggtctatgaaccctttcttccctgggcgcttgacgttggtaagaggctcggcatcacctgcgctgcgtttttcacgcagtcttgtgctgtggatgccatatatagccatgtgagagacgggaagctgtcactgccgacggaggaggccgttgagctgcccggattgcctcgccttgagcccggcgatctgccgtcctactttaccgaccctgtccctggcccttatcaggcctattttgagatgctcgtacagcaattcagtaacttggaacgagctgacatcgtcctcattaactctttctatgaactcgaaattcgggaactagattggttgaagacctcctggccagtgacaacagttgggccgacagtcccatctaagtatctagacaatgagatcccatccgacggtcactacggcttcaacctattcacaccggacataggcccctgcatgtcatggctagacttgcagacccccaaatctgtcgtctatgtctcatatggtagcatgtccgacatcaattcaagacagactgaggagatagccagagctcttcaaaattctggcaagaatttcctctgggttgtgcgaaaaacggagatgaacaaggtcccggtcgacttcgtaaatgagacttccaatcaggggttggtggtgtcatggagcccccaactagaggtgttggctcatccatcagtcggctgcttcgtgactcattgtggttggaattcgacgacagaggggttatcccttggagtgccaatgattggagtgccgcagtggacagaccaacaaaccaatgcaaagtatgttgaggacgtgtggaaggtcggggtaagggtgaaggtggatgagaagagatttctgaaaagggatgagctagagaggtgcataagagaggtgatggaaggagatatgagcgaggagataaggaagaatgcaggaaagtggagggagatggcgaaggctgcagtgagcaagggtgggagctcgaataagaacataattgaattcattgagaagtattgctcgtga

SEQ ID NO: 18 - protein, 454 aa, tulip pistil
MVVQGSHVLIVPYPGQGHLNPMLQFGKHLAHHGLTVTVATTRYILSTNPPDPKLTHITFAPISDGFDDGGFGACGDVTSYLARMESVGSQSLTDLIESSSAEGRPVRVMVYEPFLPWALDVGKRLGITCAAFFTQSCAVDAIYSHVRDGKLSLPTEEAVELPGLPRLEPGDLPSYFTDPVPGPYQAYFEMLVQQFSNLERADIVLINSFYELEIRELDWLKTSWPVTTVGPTVPSKYLDNEIPSDGHYGFNLFTPDIGPCMSWLDLQTPKSVVYVSYGSMSDINSRQTEEIARALQNSGKNFLWVVRKTEMNKVPVDFVNETSNQGLVVSWSPQLEVLAHPSVGCFVTHCGWNSTTEGLSLGVPMIGVPQWTDQQTNAKYVEDVWKVGVRVKVDEKRFLKRDELERCIREVMEGDMSEEIRKNAGKWREMAKAAVSKGGSSNKNIIEFIEKYCS

SEQ ID NO: 19 - DNA, 1365 bp, tulip pistil
atggttgtccagggaagccatgtcctcatcgtcccctaccccggccagggtcacctaaaccctatgctgcagttcggcaagcacctcgcacaccacggcctcactgtcaccgtcgccaccacccgttacatcctctccactaaccctcccgacaccaaactcagccacataaccttcgcccccatttcagacggctttgacgatggcggctttggcgcctgcggggacgtcacgtcgtacctcgcgcggatggagtcggtcgggtcccaatccttgacagacctcattgagtcaagcagtgcagagggccgccccgtccgggtaatggtctatgaaccatttcttccctgggcgcttgacgttggtaagaggctcggcatcacctgcgctgcctttttcacgcagtcttgtgctgtggatgctatatacagccatgtgagagatgggaagctttcattgccggcggaggaggcggttgagctgcccggattgcctcgtcttgagcccaacgatctcccgtcctacttcactgaccctgtcccgggcccctatcaagcttactttgagatgctcgtacaacagttgagtaatttggaacgggctgacatcgtgctcattaactctttctatgaactcgaaatccgggaactagattggctgaagacctcctggccagtaacaacagttgggcctacagtcccatctaagtatctagacaatgagatcccatccgacggtcactacggcttcaacctattcacaccagacacaggcccctgcatgtcatggctagactcgcagacccccaactccgtcgtctacgtctcatatggtagcatgtccgacatcaattcaaagcaaaccgaggagatagctagagcacttcagaactcgggcaagaatttcctttgggttgtccgaaaaacggagatgaacaaggtcccgattgacttcataaatgagacagccaatcagggactagtggtatcatggagcccccaactagaggtgttggctcatccatcagtcggctgctttgtgacccattgtggttggaattcgacgacagaagggttatcccttggagtgccaatgattggagtgccacagtggacagaccaacaaaccaatgcaaagtatgttgaggatgtgtggaaggtcgggctaagagtgaaggtggatgagaagaggtttctaaagagggatgagctagagaggtgcataagagaggtgatggaaggagataagagcgaagagataaggaagaacgcaggaaagtggagggagatggcaaagattgcagtgagcaagggtggaagctcaaataagaacatacttgaattcattgagaagtattgctcgtga

SEQ ID NO: 20 - protein, 454 aa, tulip pistil
MVVQGSHVLIVPYPGQGHLNPMLQFGKHLAHHGLTVTVATTRYILSTNPPDTKLSHITFAPISDGFDDGGFGACGDVTSYLARMESVGSQSLTDLIESSSAEGRPVRVMVYEPFLPWALDVGKRLGITCAAFFTQSCAVDAIYSHVRDGKLSLPAEEAVELPGLPRLEPNDLPSYFTDPVPGPYQAYFEMLVQQLSNLERADIVLINSFYELEIRELDWLKTSWPVTTVGPTVPSKYLDNEIPSDGHYGFNLFTPDTGPCMSWLDSQTPNSVVYVSYGSMSDINSKQTEEIARALQNSGKNFLWVVRKTEMNKVPIDFINETANQGLVVSWSPQLEVLAHPSVGCFVTHCGWNSTTEGLSLGVPMIGVPQWTDQQTNAKYVEDVWKVGLRVKVDEKRFLKRDELERCIREVMEGDKSEEIRKNAGKWREMAKIAVSKGGSSNKNILEFIEKYCS

SEQ ID NO: 21 - DNA, 1380 bp, Alstroemeria
atggctggagatggtgagcagcaacaaattaagggccatgctctcctgctcccgtatccggggcaaggccactacaaccccatgctccagttcgccaagcgcctctccttccacggcgtcgccaccaccgtcgccgtcacccgcttcatcctcagcaagaccacgccaaccccaacccccaccaacccacccatctccatcgccgccgtctccgacggctacgacgccaacgggttcggcgacgccccctccatctccgcctacctccacagcctcgagatagccggctccgccaccctcgctgacgttctcaactcccgccccgacatcaacatcctcatctacgacgccttcctcccgtgggcgctggacgtcgggaagcggctcggggtcgcgtgcgtcgctttcttcacgcagtcgtgcgcggtggacgttttgtaccgtcacgtgaacgcggggcggctggcgttgccggcgctggaggccgtgtcgctgccggggctgccggagacgattgagccgggggacttgccgtccttcttggtggacccgccgccggggccgtaccaggcatatttggagatggtgctgggtcagtacccgaacctggggggcgccgacgccgtgctcgtcaattcattctacgagctcgaaacaagggaggtggactttctgaacacagaattgccagttttgacgatcggaccaacgctcccgtcctcgtacacggacaatcgcctgcccgccgacaccgcatacggcttcaacctcttcactcccgaccccaaatcctgcatcaaatggctcgactccaagccacccaaatccgtcgtctacgtcgcgttcggtagcatggcgtcgctcggaaaggagcacgtcgccgagatagcttggtccctcaagacgggcagccacgactatatctgggtgatgaggacatcggagacgagcaagatccccaaagagtgcatcgaagatgaaactgatagagggctcatcgtcacctggagccctcagctcgaggtcttggcgcacaaggcggtcgggtgcttcgtgacgcactgcgggtggaactccaccatggaggcgattacccttggtgtgccgacggtgacgatgccgcagtggacggaccagaccacaaatgccaagtacatggaggatgtgtgggagacgggggtgaggtccaaggccgacgagaatggtgttgtgacgagggaggagattgcgaggtgcatcggggaggtgatggagggggagaggagtgagattattaggaagaatgcggagaagtggaaggaggcggccaaggtggcgatcagtgaaggcgggagctcggacaagaacatatctgaactgattagcaggtttgtcgtgtga

SEQ ID NO: 22 - protein, 459 aa, Alstroemeria
MAGDGEQQQIKGHALLLPYPGQGHYNPMLQFAKRLSFHGVATTVAVTRFILSKTTPTPTPTNPPISIAAVSDGYDANGFGDAPSISAYLHSLEIAGSATLADVLNSRPDINILIYDAFLPWALDVGKRLGVACVAFFTQSCAVDVLYRHVNAGRLALPALEAVSLPGLPETIEPGDLPSFLVDPPPGPYQAYLEMVLGQYPNLGGADAVLVNSFYELETREVDFLNTELPVLTIGPTLPSSYTDNRLPADTAYGFNLFTPDPKSCIKWLDSKPPKSVVYVAFGSMASLGKEHVAEIAWSLKTGSHDYIWVMRTSETSKIPKECIEDETDRGLIVTWSPQLEVLAHKAVGCFVTHCGWNSTMEAITLGVPTVTMPQWTDQTTNAKYMEDVWETGVRSKADENGVVTREEIARCIGEVMEGERSEIIRKNAEKWKEAAKVAISEGGSSDKNISELISRFVV

SEQ ID NO: 23 - DNA, 1380 bp, Alstroemeria
atggacggaggcgggcagcagcagcagcagcggcggagaggccacgccctcctcctcccatacccgagccaaggccacatcaaccccatgctccagttcgccaagcgcctctcctcccacggcgtcgccactaccctcgccgtcacccgcttcatcctcagcaagaccaccccaacccccgccaccccgcccgtctccatcgcccccatctccgacggctacgacgccaacggcttcgccgacgccccatccatcgccgcctacctcgccagcctcgagtccgtcggctccgccaccctcgccgacgtcctcacctcccaccccggcatcaacatcctcgtctacgaccccttcctgccgtgggtgctggatgtcgggaagcgggcaggggtcgcatgcgtcgctttcttcacgcagtcctgcgcggtggatgttgtctattgccatgtggcagcggggcggctggcgttgccggcgctggaggctgtgtcgttgccggggctgccggcgacgatggagccgggggccctgccgtccttcttggtggaaccgccgccggggcagtacccggcgtacttggagatggtgctgggtcagtacagcaacattgagaacgccgacgccgtgctcgtcaattcgttctacgagctcgaaagccaggagacagactggttgcagtcatccttaccggttaagacaatcggaccaacaatcccctcctcctacatagacaaccgaataccgaccgactcctcatatggcttcaacctcttcacccccgaccccaaatcctgcaccaaatggctcgactccaagccaccacaatccgtcgtctacgtctcgttcggcagcatggggtcgctcggaacggagcagattgtcgagatagcttggtgcctcaagacgagcaaccacaactatctttgggtggttagggcttcagaatcaagcaagatccccgaagagtacatcgaagatgaaaatgatagaaggctcattgtcacctggagccctcagcttgaggtcttggctcacaaggcggtcgggtgcttcgtgatgcattgcgggtggaactcaaccatggaggcgattagctttggtgtgcccatggtggccgtgccgcaatggtcggaccaaactacgaactcgaagtatgtggaggacatttgggggattggggttagggcaaagtttgatgagaatgatgttgtggagaggaaagagattgcgaggtgcatcggagaggtgatggaaggggagaggagtgagattatgaggaagaatgcggagaagtggaaggaggcggctaaggtggcaattagtgaaggcgggagctcggataaaaacatagttgagtttattagtaggtttgtcttgtaa

SEQ ID NO: 24 - protein, 459 aa, Alstroemeria
MDGGGQQQQQRRRGHALLLPYPSQGHINPMLQFAKRLSSHGVATTLAVTRFILSKTTPTPATPPVSIAPISDGYDANGFADAPSIAAYLASLESVGSATLADVLTSHPGINILVYDPFLPWVLDVGKRAGVACVAFFTQSCAVDVVYCHVAAGRLALPALEAVSLPGLPATMEPGALPSFLVEPPPGQYPAYLEMVLGQYSNIENADAVLVNSFYELESQETDWLQSSLPVKTIGPTIPSSYIDNRIPTDSSYGFNLFTPDPKSCTKWLDSKPPQSVVYVSFGSMGSLGTEQIVEIAWCLKTSNHNYLWVVRASESSKIPEEYIEDENDRRLIVTWSPQLEVLAHKAVGCFVMHCGWNSTMEAISFGVPMVAVPQWSDQTTNSKYVEDIWGIGVRAKFDENDVVERKEIARCIGEVMEGERSEIMRKNAEKWKEAAKVAISEGGSSDKNIVEFISRFVL

SEQ ID NO: 25 - DNA, 17 bp, Artificial Sequence
  M13 Forward (-20) Primer
gtaaaacgacggccagt

SEQ ID NO: 26 - DNA, 19 bp, Artificial Sequence
  M13 Reverse Primer
ggaaacagctatgaccatg

SEQ ID NO: 27 - DNA, 20 bp, Artificial Sequence
  Primer NW5
gataaccacacggaggacag

SEQ ID NO: 28 - DNA, 20 bp, Artificial Sequence
  Primer NW6
caggaatgatccatccgaac

SEQ ID NO: 29 - DNA, 20 bp, Artificial Sequence
  Primer NW7
ttctccacagccgtaacatg

SEQ ID NO: 30 - DNA, 894 bp, Alstroemeria
gagaataccatatgtgttgctgctatcttgggctcaacccttactggagagttcgaagatgttaaactactgaacaaacttcttgaagaaaagaacaaggaaactgggtgggacacacccattcatgttgatgctgctagtggtggattcattgctccttttctatacccagaactggaatgggatttccgattaccactggtgaagagtattaatgtcagcggacacaaatatggccttgtttatgcaggtgtgggttgggttgtctggaggaacaaagaggatcttcccgaagagctcattttccatataaactaccttggggcagatcagcctactttcaccctcaatttctcaaaaggttcaagccagataattgctcaatattatcaattcattcgccttggttttcaggggtataagaacataatggaaaactgcatggagaacacaagaatactgagagaaggtctgcaggagacgggccgtttcgagatagtctccaaagatattggggtgcctcttgttgcatttgctctcaaggacagcagccagcacactgtctttgagatagcggacaccatgagaaggttcggatggatcattcctgcatacaccatgccaaaggacgcggagcacatagctgtcctacgcgtggttatcagggaggatttcagcaggagccttgctgagcgcctagttaatgacatgaagaaggtgctggctgagctggacgtacttcccagtcgcatcaccaccattgccgatgttacggctgtggagaacgataatggcgaagctgtgatcaagaagagtttcctggagatagagaagaaggttattacacattggaaggatgtagtgatgaacggcaagaagactaataaagtctgc

SEQ ID NO: 31 - DNA, 298 bp, Alstroemeria
ggacaattcctgcatacacaatgccaaaggacgttgagcacatagccgtccttcgtgtggttatcagggaggacttcagccggagcctcgccgagcgcctagctaatgacatgaagaaggtgttggttgagctggacatacatcccagtcgcaccaccaccattgcccacgttaaggcagtggagaatggcaatggcaattatgtgatcaagaagagtattgtagagaatggcaatggcgaacatgtgatcaagaagagcattggggagaatggcaatggcaaacacgcgatcaagaa

SEQ ID NO: 32 - DNA, 20 bp, Artificial Sequence
  Primer NW12
cattgtgtatgcaggaattg

SEQ ID NO: 33 - DNA, 20 bp, Artificial Sequence
  Primer NW13
cggcgaggctccggctgaag

SEQ ID NO: 34 - DNA, 20 bp, Artificial Sequence
  Primer NW14
ttcttgatcacatgttcgcc

SEQ ID NO: 35 - DNA, 21 bp, Artificial Sequence
  Primer NW15
ttgcaatcactgcatccctgc

SEQ ID NO: 36 - DNA, 20 bp, Artificial Sequence
  Primer pBluescript T3 (from Stratagene Cloning Systems, La Jolla,CA)
aattaaccctcactaaaggg

SEQ ID NO: 37 - DNA, 19 bp, Artificial Sequence
  Primer NW18
ctctttaatgcgccaattg

SEQ ID NO: 38 - DNA, 19 bp, Artificial Sequence
  Primer NW19
tcttgcattctccgtgcag

SEQ ID NO: 39 - DNA, 20 bp, Artificial Sequence
  Primer NW20
cggatccccggggtccgtac

SEQ ID NO: 40 - DNA, 21 bp, Artificial Sequence
  Primer NW21
tcatgccgcgtttgcaatcac

SEQ ID NO: 41 - DNA, 20 bp, Artificial Sequence
  Primer NW22
aatttactggcgctcatgcc

SEQ ID NO: 42 - DNA, 20 bp, Artificial Sequence
  Primer NW23
attcctgcatacacaatgcc

SEQ ID NO: 43 - DNA, 33 bp, Artificial Sequence
  Primer NW9
cccaagcttttatcagcaaactttattagtctt

SEQ ID NO: 44 - DNA, 27 bp, Artificial Sequence
  Primer NW10
catgccatggttctctccagcgccgtc

SEQ ID NO: 45 - DNA, 29 bp, Artificial Sequence
  Primer NW24
ggactagtatggctctctccagcgtcgtc

SEQ ID NO: 46 - DNA, 33 bp, Artificial Sequence
  Primer NW25
cccaagcttttatcatgccgcgtttgcaatcac

SEQ ID NO: 47 - DNA, 24 bp, Artificial Sequence
  Primer E20-5
atgatgccgctggcctgctccgat

SEQ ID NO: 48 - DNA, 24 bp, Artificial Sequence
  Primer E20-3
ctttgctactgctatttctgagtc

SEQ ID NO: 49 - DNA, 24 bp, Artificial Sequence
  Primer C16-5
atgatgatcgccggcaagctcctc

SEQ ID NO: 50 - DNA, 24 bp, Artificial Sequence
  Primer C16-3
ctgagattttaactccttcaccct

SEQ ID NO: 51 - DNA, 24 bp, Artificial Sequence
  Primer gabT-5
atgaacagcaataaagagttaatg

SEQ ID NO: 52 - DNA, 24 bp, Artificial Sequence
  Primer gabT-3
ctcctgcttcgcctcatcaaaaca

SEQ ID NO: 53 - DNA, 17 bp, Artificial Sequence
  Primer ETP5
atggaggtgggattcct

SEQ ID NO: 54 - DNA, 19 bp, Artificial Sequence
  Primer ETP3
ctacgatgcatctccatac

SEQ ID NO: 55 - DNA, 19 bp, Artificial Sequence
  Primer ADS5
atggaagtagggtttctgg

SEQ ID NO: 56 - DNA, 19 bp, Artificial Sequence
  Primer ADS3
ctattcgcgggagaatttc

SEQ ID NO: 57 - DNA, 21 bp, Artificial Sequence
  Primer pTrcHisforward
gaggtatatattaatgtatcg

SEQ ID NO: 58 - DNA, 18 bp, Artificial Sequence
  Primer pTrcHisreverse
gatttaatctgtatcagg

SEQ ID NO: 59 - DNA, 24 bp, Artificial Sequence
  Primer N21-5
atggttgtccagggaagccatgtc

SEQ ID NO: 60 - DNA, 24 bp, Artificial Sequence
  Primer N21-3His
tcccgagcaatacttctcaatgaa

SEQ ID NO: 61 - DNA, 24 bp, Artificial Sequence
  Primer L14-5Pag
tcatggttgtccagggaagccatg

SEQ ID NO: 62 - DNA, 24 bp, Artificial Sequence
  Primer L14-3Xho
gagaagtattgctcgggactcgag

SEQ ID NO: 63 - DNA, 24 bp, Artificial Sequence
  Primer K7-5Pag
tcatggctggagatggtgagcagc

SEQ ID NO: 64 - DNA, 24 bp, Artificial Sequence
  Primer K7-3Hind
agcaggtttgtcgtgggaaagctt

SEQ ID NO: 65 - DNA, 24 bp, Artificial Sequence
  Primer E12-5Pag
tcatggacggaggcgggcagcagc

SEQ ID NO: 66 - DNA, 24 bp, Artificial Sequence
  Primer E12-3Hind
agtaggtttgtcttgtcaaagctt

SEQ ID NO: 67 - protein, 10 aa, Artificial Sequence
  WO 02/00904
ELVISLIVES

(19) United States
(12) Patent Application Publication
(10) Pub. No.: US 2003/0170653 A1
(43) Pub. Date: Sep. 11, 2003

(54) Biological method for the production of tuliposide A and its intermediates

(76) Inventors: Howard Glenn Damude, Hockessin, DE (US); Vikram Prabhu, Wilmington, DE (US); Hong Wang, Kennett Square, PA (US); Dennis H. Flint, Newark, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/167,547

(22) Filed: Jun. 10, 2002

Related U.S. Application Data
(60) Provisional application No. 60/297,198, filed on Jun. 8, 2001.

(51) Int. Cl. C12Q 1/68; C07H 21/04; C12N 9/24; C12P 21/02; C12N 1/21; C12N 5/04; C07C 59/147

(52) U.S. Cl. 435/6; 435/69.1; 435/200; 435/320.1; 435/252.3; 435/419; 536/23.2; 562/577

(57) ABSTRACT

This invention relates to genes encoding key enzymes in the biosynthesis of α-methylene-γ-butyrolactone (tulipalin A). The key enzymes are glutamate decarboxylase, γ-aminobutyrate aminotransferase, γ-hydroxybutyrate dehydrogenase and UDP-glucosyltransferase. The genes and their expression products are useful for the creation of recombinant organisms that have the ability to synthesize tulipalin A, tuliposide A or any tuliposide A pathway intermediates.

### FIELD OF THE INVENTION

The invention relates to the field of plant molecular biology and biochemistry. More specifically, the invention relates to the cloning of genes that encode proteins involved in the biosynthesis of α-methylene-γ-butyrolactone (tulipalin A) and its intermediates.

### BACKGROUND OF THE INVENTION

α-Methylene-γ-butyrolactone, hereinafter referred to as tulipalin A, is a natural product produced by plants of the Alstroemeriaceae and Liliaceae families and has antimicrobial, fungicidal and insecticidal properties (Cavalito and Haskell, J. Am. Chem. Soc. 68:2332-2334 (1946); Bergman et al., Recueil. Trav. Chim. Pays-Bas. 86:709-714 (1967); Brongersma-Oosterhoff, Recueil. Trav. Chim. Pays-Bas. 86:705-708 (1967); Tschesche et al., Chem. Ber. 102:2057-2071 (1969); Slob, Phytochemistry 12:811-815 (1973); Kim et al., Biosci. Biotechnol. Biochem. 62:1546-1549 (1998)).

Tuliposide A exists as a glucoside in plants and is believed to be stored in the vacuoles of the plant cells. It is found to be present in large quantities (0.2-2% w/w fresh plants) in all parts of the plant. Moreover, tuliposide A is made at concentrations as high as 30% dry weight in tulip pistils and 10% dry weight in the leaves of Alstroemeria and tulip (Slob et al., Phytochemistry 14:1997-2005 (1975)). Following damage to the plant, the glucose from tuliposide A is hydrolyzed and the open chain form of tulipalin A (α-methylene-γ-hydroxybutyrate) is cyclized to form the lactone tulipalin A.

In addition to its antimicrobial, fungicidal and insecticidal properties, tulipalin A is also a known skin irritant that causes an allergic response to persons who frequently handle plant tissue containing high levels of the compound, particularly those working with Alstroemeria and tulips (Rook, Contact Dermatitis 7:355-356 (1981)). Typically, patients are florists who exhibit hyperkeratosis, fissuring, erythema and tenderness of the tips of all digits on both hands. The ailment is commonly referred to as “tulip finger” (Verrspyck, J. Dermatology 81:737-745 (1969)). Since certain species of Alstroemeria are becoming increasingly popular with amateur gardeners, several cases of allergic contact dermatitis have been seen among those not associated with the floral industry.

Many plants that synthesize tulipalin A also make other compounds that contain an exo-double bond on a carbon atom adjacent to a carboxyl group. These include γ-methyleneglutamate and γ-methyleneglutamine (Zacharius et al., J. Amer. Chem. Soc. 76:1961 (1954); Fowden and Steward, Ann. Bot., Lond., N.S. 21:53-67 (1957)). Other compounds related in structure to γ-methyleneglutamate, such as γ-hydroxy-γ-methylglutamate, α-keto-γ-methyleneglutarate and γ-methylglutamate, have also been identified in tulipalin A-producing plants (Fowden and Steward, Ann. Bot. Lond., N.S. 21:53-67 (1957); Ohyama et al., Soil Sci. Plant Nutr. 34:75-86 (1988)). These glutamate analogs have also been identified in many other plant species along with other structurally related metabolites such as α-keto-γ-hydroxy-γ-methylglutarate, γ-ethylideneglutamate, γ-propylideneglutamate, γ-ethyl-γ-hydroxyglutamate, γ-hydroxyglutamate, γ-ethylglutamate, γ-hydroxymethylglutamate, β-hydroxy-γ-methylglutamate and β-hydroxy-γ-methyleneglutamate.

Tulipalin A is an important monomer in various thermoplastic applications having desired characteristics. Thermoplastics comprise a large body of commercially important products. Consequently, tulipalin A has been the subject of intensive chemical synthetic studies. Among the uses of thermoplastics are those in which the optical properties of the polymer are important, particularly when the polymer is an optically clear material with little distortion of optical images. Currently, the cost of producing tulipalin A is too high to warrant commercial production of the resulting polymers. Some of the current synthetic routes suffer from low yields, by-product formation and expensive starting materials. A biosynthetic pathway presents an alternate route to the production of this important monomer.

Little is known about the biosynthesis of tulipalin A. In fact, the pathway to tulipalin A has not previously been elucidated. None of the genes involved have been identified or cloned. None of the biosynthetic enzymes have been characterized or purified. One labeling experiment carried out with ¹⁴C-pyruvate in tulips suggested that tulipalin A was made initially from the condensation of pyruvate and acetyl-Coenzyme A (Hutchinson et al., Chem. Comm. 18:1189 (1970)). The authors of this paper ruled out an initial condensation of two pyruvate molecules. A few papers have been published which address the question of biosynthesis of γ-hydroxy-γ-methylglutamate and γ-methyleneglutamate in various plants. For these metabolites, a pathway initially involving the condensation of two pyruvate molecules followed by transamination was proposed, but the data was inconclusive (Fowden et al., Annals of Botany, N.S. 22:1958 (1958); Linko et al., Acta Chemica Scandinaviaca 12:68 (1958)). Other research disputes the direct incorporation of two pyruvate molecules (Shannon et al., J. Biol. Chem. 261:3342 (1962); Marcus et al., J. Biol. Chem. 261:3348 (1962); Peterson et al., Phytochemistry 11:663 (1972)), although condensation of one pyruvate with another molecule could not be ruled out. An alternate pathway involving oxidation of leucine to γ-methylglutamate, followed by further oxidization to γ-hydroxy-γ-methylglutamate has also been proposed but no evidence was found for further conversion to γ-methyleneglutamate.

The instant invention has identified the biosynthetic pathway to tulipalin A and has cloned many of the genes (γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase) involved. Genes encoding glutamate decarboxylases, γ-aminobutyrate aminotransferases, γ-hydroxybutyrate dehydrogenases and UDP-glucosyl transferases from plant sources have been described (WO 00/61763; Baum et al., J. Biol. Chem. 268:19610 (1993); Vogt et al., Trends Plant Science 5(9):380-386 (2000)). However, these plant sources have not been shown to synthesize tulipalin A. Glutamate decarboxylase from Escherichia coli, Capsicum sp., barley, tulip and Clostridium welchii have all been shown to accept γ-methyleneglutamate as a substrate, although they are more specific for glutamate than for 4-methyleneglutamate (Sukhareva et al., Moleckulyamaya Biologiya 11:394 (1977); Fowden, J. Exp. Bot. 5:28 (1954)).

The problem to be solved, therefore, was to elucidate the biosynthetic pathway to tulipalin A and clone the genes involved in order to: 1.) provide an alternative synthetic route for the production of tulipalin A as a monomer source for thermoplastic applications; and, 2.) engineer plants having decreased tulipalin A biological activity. Applicants have solved the problem by identifying and characterizing a biosynthetic pathway to tulipalin A and by cloning the genes responsible for biosynthesis of tulipalin A. Furthermore, proteins made by expressing each of the tulipalin A biosynthetic genes in recombinant Escherichia coli or yeast hosts were able to catalyze the correct step in tulipalin A biosynthesis. The instant invention has overcome the problems encountered during chemical synthesis of tulipalin A by providing an alternative biosynthetic pathway for its production.

### SUMMARY OF THE INVENTION

The present invention provides an isolated nucleic acid fragment encoding a tuliposide A synthesizing protein selected from the group consisting of: (a) an isolated nucleic acid molecule encoding the amino acid sequence as set forth in SEQ ID NO: 2, SEQ ID NO: 4, SEQ ID NO: 6, SEQ ID NO: 8, SEQ ID NO: 10, SEQ ID NO: 14, SEQ ID NO: 18, SEQ ID NO: 20, SEQ ID NO: 22 and SEQ ID NO: 24; (b) an isolated nucleic acid molecule that hybridizes with (a) under the following hybridization conditions: 0.1× SSC, 0.1% SDS at 65° C., and washed with 2× SSC, 0.1% SDS followed by 0.1× SSC, 0.1% SDS; and (c) an isolated nucleic acid molecule that is completely complementary to (a) or (b).

The invention also provides chimeric genes comprised of the instant nucleic acid fragments and suitable regulatory sequences as well as the polypeptides encoded by said sequences.

Additionally, the invention provides recombinant organisms transformed with the chimeric genes of the instant invention.

The invention further provides methods for obtaining all or a portion of the instant sequences by either primer-directed amplification protocols or by hybridization techniques using primers or probes derived from the instant sequences.

In another embodiment the invention provides a mutated microbial gene encoding a protein having an altered biological activity produced by a method comprising the steps of (i) digesting a mixture of nucleotide sequences with restriction endonucleases wherein said mixture comprises:

a) a native microbial gene selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;

b) a first population of nucleotide fragments which will hybridize to the native microbial sequence;

c) a second population of nucleotide fragments which will not hybridize to the native microbial sequence;

wherein a mixture of restriction fragments are produced; (ii) denaturing the mixture of restriction fragments; (iii) incubating the denatured said mixture of restriction fragments of step (ii) with a polymerase; and (iv) repeating steps (ii) and (iii) wherein a mutated microbial gene is produced encoding a protein having an altered biological activity.

The invention further provides a bioprocess for converting γ-methyleneglutamate to tuliposide A comprising: contacting a transformed host cell under suitable growth conditions with an effective amount of γ-methyleneglutamate whereby tuliposide A is produced, the transformed host cell comprises a tuliposide A synthesizing protein selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 and mixtures thereof, under the control of suitable regulatory sequences.

Further embodiments of the invention use an enzyme catalyst having the activity of a tuliposide A synthesizing protein in the form of whole microbial cells, permeabilized microbial cells, one or more cell components of a microbial cell extract, and partially purified enzyme(s), or purified enzyme(s). In any form and optionally, the enzyme catalyst may be immobilized in or on a soluble or insoluble support.

### BRIEF DESCRIPTION OF THE DRAWINGS AND SEQUENCE DESCRIPTIONS

FIG. 1 shows the proposed biosynthetic pathway to tulipalin A.

FIG. 2 shows the ¹³C-labeling NMR studies for the biosynthesis of tulipalin A.

The invention can be more fully understood from the following detailed description, the figures, and the accompanying sequence descriptions that form a part of this application.

The following sequence descriptions and sequences listings attached hereto comply with the rules governing nucleotide and/or amino acid sequence disclosures in patent applications as set forth in 37 C.F.R. §1.821-1.825 (“Requirements for Patent Applications Containing Nucleotide Sequences and/or Amino Acid Sequence Disclosures—The Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NOs: 1-24 are full length genes or proteins as identified in Table 1. Peptide sequences were derived from the respective nucleic acid sequence.

TABLE 1
Summary of Gene and Protein SEQ ID Numbers

| | | | SEQ |
|---|---|---|---|
| | | SEQ ID | ID |
| | | Nucleic | Pep- |
| Description | Organism | acid | tide |
| gad2 | Alstroemeria | 1 | 2 |
| (glutamate decarboxylase homolog) | | | |
| gad3del | Alstroemeria | 3 | 4 |
| (glutamate decarboxylase homolog) | | | |
| gad3chim | Alstroemeria | 5 | 6 |
| (glutamate decarboxylase homolog) | | | |
| e20 | tulip pistil | 7 | 8 |
| (y-aminobutyrate aminotransferase | | | |
| homolog) | | | |
| c16 | Alstroemeria | 9 | 10 |
| (γ-aminobutyrate aminotransferase | | | |
| homolog) | | | |
| gabT gene | Escherichia coli | 11 | 12 |
| (γ-aminobutyrate aminotransferase) | | | |
| ghbd1 | tulip pistil | 13 | 14 |
| (γ-hydroxybutyrate dehydrogenase | | | |
| homolog) | | | |
| ghbd2 | Arabidopsis | 15 | 16 |
| (γ-hydroxybutyrate dehydrogenase | | | |
| homolog) | | | |
| n21 | tulip pistil | 17 | 18 |
| (UDP-glucosyltransferase homolog) | | | |
| I14 | tulip pistil | 19 | 20 |
| (UDP-glucosyltransferase homolog) | | | |
| k7 | Alstroemeria | 21 | 22 |
| (UDP-glucosyltransferase homolog) | | | |
| e12 | Alstroemeria | 23 | 24 |
| (UDP-glucosyltransferase homolog) | | | |

SEQ ID NOs: 25 and 26 are those of the M13 forward (−20) and reverse primers, respectively.

SEQ ID NOs: 27, 28, and 29 are primers NW5, NW6, and NW7, respectively.

SEQ ID NO: 30 is the nucleotide sequence of gad1 encoding a glutamate decarboxylase homolog (from Alstroemeria).

SEQ ID NO: 31 is the nucleotide sequence of gad3 encoding a glutamate decarboxylase homolog (from Alstroemeria).

SEQ ID NOs: 32-35 are the primers NW12, NW13, NW14, and NW15, respectively.

SEQ ID NO: 36 is a pBluescript T3 primer (Stratagene).

SEQ ID NOs: 37-46 are the primers NW18, NW19, NW20, NW21, NW22, NW23, NW9, NW10, NW24, and NW25, respectively.

SEQ ID NOs: 47 and 48 are the primers E20-5 and E20-3, respectively.

SEQ ID NOs: 49 and 50 are the primers C16-5 and C16-3, respectively.

SEQ ID NO: 51 and 52 are the primers gabT-5 and gabT-3, respectively.

SEQ ID NO: 53 and 54 are the primers ETP5 and ETP3, respectively.

SEQ ID NO: 55 and 56 are the primers ADS5 and ADS3, respectively.

SEQ ID NO: 57 and 58 are the primers pTrcHis forward and pTrcHis reverse, respectively.

SEQ ID NO: 59 and 60 are the primers N21-5 and N21-3His, respectively.

SEQ ID NO: 61 and 62 are the primers L14-5Pag and L14-3Xho, respectively.

SEQ ID NO: 63 and 64 are the primers K7-5Pag and K7-3Hind, respectively.

SEQ ID NO: 65 and 66 are the primers E12-5Pag and E12-3Hind, respectively.

SEQ ID NO: 67 is the peptide sequence ELVISLIVES, for use in gene cosuppression, as disclosed in WO 02/00904.

### DETAILED DESCRIPTION OF THE INVENTION

The instant invention provides genes encoding key enzymes in the biosynthesis of tulipalin A. The key enzymes are γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase. The genes and their expression products are useful for the creation of recombinant organisms that have the ability to synthesize tulipalin A, tuliposide A or any of their pathway intermediates.

Tuliposide A pathway intermediates (γ-methyleneglutamate, α-methylene-γ-aminobutyrate, α-methylenesuccinate semialdehyde and α-methylene-γ-hydroxybutyrate) may also have utility in many applications.

The genes involved in tulipalin A biosynthesis were isolated herein from Alstroemeria caryophylla. Each gene has been expressed in Escherichia coli or yeast and the protein encoded by each gene has been shown to catalyze a reaction in each step of the tulipalin A pathway. Homologs of some of these genes that encode proteins having similar functions were also isolated from Tulipa gesneriana. Previous studies on the biosynthesis of tulipalin A used ¹⁴C-labeled pyruvate in feeding experiments in tulips (Hutchinson et al., Chem. Comm. 18:1189 (1970)). From these studies it was concluded that tulipalin A was synthesized by an initial condensation of acetyl-coenzyme A with pyruvate. The inventors of the instant invention prepared and assayed enzyme extracts from the tulipalin A-producing Alstroemeria plant, but were unable to identify an activity for the condensation of acetyl-coenzyme A with pyruvate. It was therefore concluded that this step was not the “first” step in tulipalin A biosynthesis and an alternate pathway was sought.

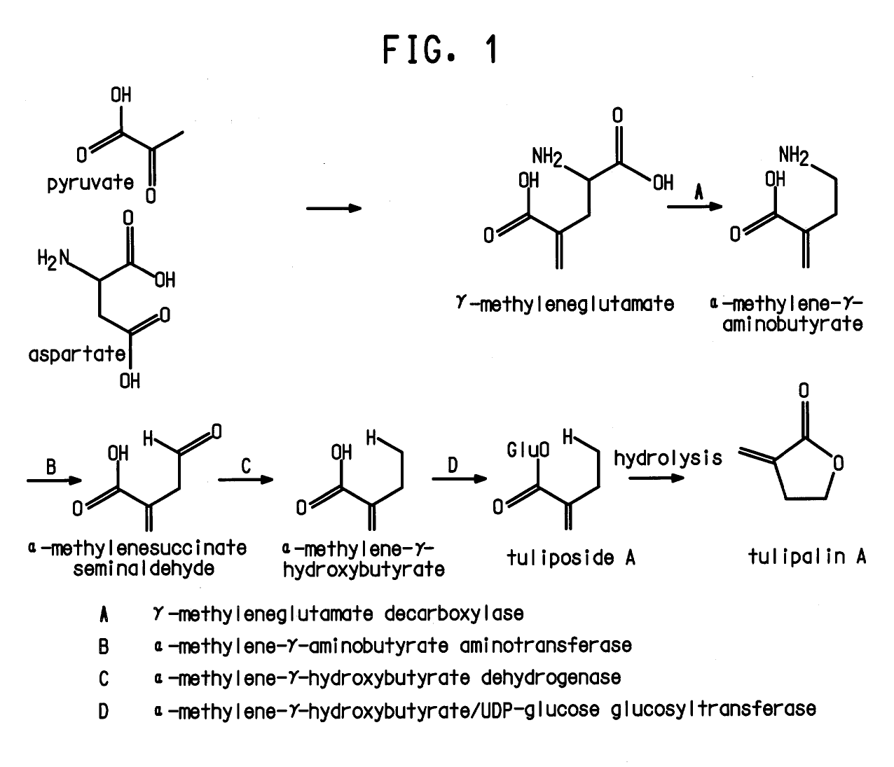

γ-Methyleneglutamate is an unusual amino acid that is structurally related to tulipalin A and occurs in relatively high levels in tulip plants. The inventors of the instant invention analyzed another tulipalin A-producing plant, Alstroemeria pulchilla, for the presence of γ-methyleneglutamate and found that this plant also makes it in relatively high levels, the first time this result has been shown. It was then proposed that γ-methyleneglutamate is an intermediate in tulipalin A biosynthesis and a new biosynthetic pathway to tulipalin A was postulated herein, as shown in FIG. 1.

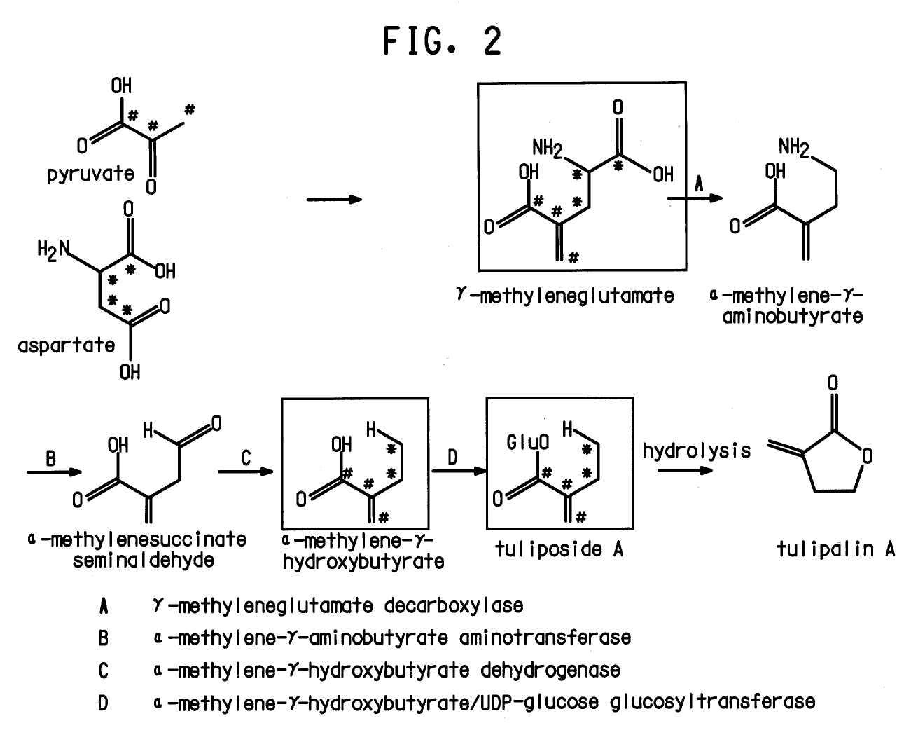

It was confirmed that this pathway was correct by feeding Alstroemeria and tulip plants various ¹³C-labeled metabolic precursors and evaluating their incorporation into tulipalin A and intermediates using nuclear magnetic resonsance (NMR), as shown in FIG. 2. First, various methods of NMR were used to identify tulipalin A, tuliposide A and γ-methyleneglutamate in Alstroemeria plants. Alstroemeria plants were then fed ¹³C-pyruvate, ¹³C-glucose, ¹³C-acetate and ¹³C-aspartate. Plant extracts were prepared at various times and then analyzed by NMR. In this way, it was determined that the same pattern of incorporating the 13C label had occurred in γ-methyleneglutamate as for tulipalin A, thus lending support to the proposed pathway. Similar experiments were then completed using ¹³C-labeled γ-methyleneglutamate and the results showed the expected labeling pattern in tulipalin A. The instant invention confirms that tulipalin A is made from pyruvate and aspartate through a γ-methyleneglutamate intermediate. Furthermore, the instant invention cloned the genes involved in each step of the pathway and revealed that they were able to catalyze the desired reaction.

Definitions

In this disclosure, a number of terms and abbreviations are used. The following definitions are provided.

“Open reading frame” is abbreviated ORF.

“Polymerase chain reaction” is abbreviated PCR.

“ATCC” refers to the American Type Culture Collection International Depository located at 10801 University Boulevard, Manassaa, Va. 20110-2209, U.S.A. The “ATCC No.” is the accession number to cultures on deposit with the ATCC.

The term “tuliposide A synthesizing protein” refers to an enzyme in the tuliposide A biosynthetic pathway. Specific examples include γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase.

The term “tuliposide A pathway intermediate” refers to a compound produced during the synthesis of tuliposide A. Specific examples include γ-methyleneglutamate, α-methylene-γ-aminobutyrate, α-methylenesuccinate semialdehyde, α-methylene-γ-hydroxybutyrate.

The term “γ-methyleneglutamate decarboxylase” refers to an enzyme that bioconverts γ-methyleneglutamate to α-methylene-γ-aminobutyrate.

The term “α-methylene-γ-aminobutyrate aminotransferase” refers to an enzyme that bioconverts α-methylene-γ-aminobutyrate to α-methylenesuccinate seminaldehyde.

The term “α-methylene-γ-hydroxybutyrate dehydrogenase” refers to an enzyme that bioconverts α-methylenesuccinate seminaldehyde to α-methylene-γ-hydroxybutyrate.

The term “α-methylene-γ-hydroxybutyrate/UDP-glucose glucosyltransferase” refers to an enzyme that bioconverts \tilde{α}methylene-γ-hydroxybutyrate to tuliposide A.

The terms “biotransformation” and “bioconversion” are used interchangeably and refer to the process of enzymatic conversion of a compound to another form or compound. The process of bio-conversion or bio-transformation is typically carried out by a biocatalyst.

“Enzyme catalyst” or “whole microbial cell catalyst” refers to a catalyst that is characterized by the activity of a tuliposide A synthesizing protein. The enzyme catalyst may be in the form of a whole microbial cell, permeabilized microbial cell(s), one or more cell components of a microbial cell extract, partially purified enzyme(s), or purified enzyme(s).

The term “isolated nucleic acid fragment” or “isolated nucleic acid molecule” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6× SSC, 0.5% SDS at room temperature for 15 min, then repeated with 2× SSC, 0.5% SDS at 45° C. for 30 min, and then repeated twice with 0.2× SSC, 0.5% SDS at 50° C. for 30 min. A more preferred set of stringent conditions uses higher temperatures in which the washes are identical to those above except for the temperature of the final two 30 min washes in 0.2× SSC, 0.5% SDS was increased to 60° C. Another preferred set of highly stringent conditions uses two final washes in 0.1× SSC, 0.1% SDS at 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment, the length for a hybridizable nucleic acid is at least about 10 nucleotides. Preferably, a minimum length for a hybridizable nucleic acid is at least about 15 nucleotides; more preferably at least about 20 nucleotides; and most preferably, the length is at least 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches partial or complete amino acid and nucleotide sequences encoding one or more particular microbial proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the glutamate decarboxylase, γ-aminobutyrate aminotransferase, γ-hydroxybutyrate dehydrogenase and UDP-glucosyltransferase enzymes as set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 and SEQ ID NO: 24. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence.

“Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites, and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065; WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. Furthermore, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “altered biological activity” herein refers to an activity associated with a protein encoded by a microbial nucleotide sequence which can be measured by an assay method, where that activity is either greater than or less than the activity associated with the native microbial sequence. “Enhanced biological activity” refers to an altered activity that is greater than that associated with the native sequence. “Diminished biological activity” is an altered activity that is less than that associated with the native sequence.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990)), DNASTAR (DNASTAR, Inc., Madison, Wis.), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, the results of the analysis will be based on the “default values” of the program referenced, unless otherwise specified. As used herein “default values” will mean any set of values or parameters which originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

Identification of Enzymes and Homologs

A variety of nucleotide sequences have been isolated from Alstroemeria, Tulipa gesneriana (tulip pistal), and Escherichia coli encoding gene products of tuliposide A synthesizing proteins. The present invention provides examples of γ-methyleneglutamate decarboxylase, α-methylene-γ-aminobutyrate aminotransferase, α-methylene-γ-hydroxybutyrate dehydrogenase, and α-methylen\tilde{e}γ-hydroxybutyrate/UDP-glucose glucosyltransferase genes and gene products having the ability to bioconvert γ-methyleneglutamate to α-methylene-γ-aminobutyrate to α-methylenesuccinate seminaldehyde to α-methylene-γ-hydroxybutyrate and then to tuliposide A. The nucleic acid sequences for these enzymes are set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 and SEQ ID NO: 24. It will be appreciated that other glutamate decarboxylase, γ-aminobutyrate aminotransferase, γ-hydroxybutyrate dehydrogenase and glucosyl transferase genes having similar substrate specificity may be identified and isolated on the basis of sequence-dependent protocols.

Comparison of the gad2 nucleotide base and deduced amino acid sequences to public databases reveals that the most similar known sequences range from about 78% identical to the amino acid sequence of gad2 reported herein over length of 498 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred gad2 encoding nucleic acid sequences corresponding to the instant ORF's are those encoding active proteins and which are at least 80% identical to the nucleic acid sequences of reported herein. More preferred gad2 nucleic acid fragments are at least 90% identical to the sequences herein. Most preferred are gad2 nucleic acid fragments that are at least 95% identical to the nucleic acid fragments reported herein.

Comparison of the gad3del nucleotide base and deduced amino acid sequences to public databases reveals that the most similar known sequences range from about 74% identical to the amino acid sequence of gad3del reported herein over length of 509 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred gad3del encoding nucleic acid sequences corresponding to the instant ORF's are those encoding active proteins and which are at least 80% identical to the nucleic acid sequences of reported herein. More preferred gad3del nucleic acid fragments are at least 90% identical to the sequences herein. Most preferred are gad3del nucleic acid fragments that are at least 95% identical to the nucleic acid fragments reported herein.

Comparison of the gad3chim nucleotide base and deduced amino acid sequences to public databases reveals that the most similar known sequences range from about 74% identical to the amino acid sequence of gad3chim reported herein over length of 529 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred gad3chim encoding nucleic acid sequences corresponding to the instant ORF's are those encoding active proteins and which are at least 80% identical to the nucleic acid sequences of reported herein. More preferred gad3chim nucleic acid fragments are at least 90% identical to the sequences herein. Most preferred are gad3chim nucleic acid fragments that are at least 95% identical to the nucleic acid fragments reported herein.

Comparison of the e20 nucleotide base and deduced amino acid sequences to public databases reveals that the most similar known sequences range from about 77% identical to the amino acid sequence of e20 reported herein over length of 471 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred e20 encoding nucleic acid sequences corresponding to the instant ORF's are those encoding active proteins and which are at least 80% identical to the nucleic acid sequences of reported herein. More preferred e20 nucleic acid fragments are at least 90% identical to the sequences herein. Most preferred are e20 nucleic acid fragments that are at least 95% identical to the nucleic acid fragments reported herein.

Comparison of the c16 nucleotide base and deduced amino acid sequences to public databases reveals that the most similar known sequences range from about 80% identical to the amino acid sequence of c16 reported herein over length of 507 amino acids using a BLASTP analysis (Altschul et al., J. Mol. Biol. 215:403-410 (1990)). More preferred amino acid fragments are at least about 80%-90% identical to the sequences herein. Most preferred are nucleic acid fragments that are at least 95% identical to the amino acid fragments reported herein. Similarly, preferred c16 encoding nucleic acid sequences corresponding to the instant ORF's are those encoding active proteins and which are at least 80% identical to the nucleic acid sequences of reported herein. More preferred c16 nucleic acid fragments are at least 90% identical to the sequences herein. Most preferred are c16 nucleic acid fragments that are at least 95% identical to the nucleic acid fragments reported herein.

Comparison of the ghbd1 nucleotide base and deduced amino acid sequences to public databases reveals that the most similar known sequences range from about 81 % identical to the amino acid sequence of ghbd1 reported herein over length of 290 amino acids using a BLASTP analysis (Altschul et al., J. Mol. Biol. 215:403-410 (1990)). More preferred amino acid fragments are at least about 80%-90% identical to the sequences herein. Most preferred are nucleic acid fragments that are at least 95% identical to the amino acid fragments reported herein. Similarly, preferred ghbd1 encoding nucleic acid sequences corresponding to the instant ORF's are those encoding active proteins and which are at least 80% identical to the nucleic acid sequences of reported herein. More preferred ghbd1 nucleic acid fragments are at least 90% identical to the sequences herein. Most preferred are ghbd1 nucleic acid fragments that are at least 95% identical to the nucleic acid fragments reported herein.

Comparison of the n21 nucleotide base and deduced amino acid sequences to public databases reveals that the most similar known sequences range from about 51 % identical to the amino acid sequence of n2 reported herein over length of 454 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred n21 encoding nucleic acid sequences corresponding to the instant ORF's are those encoding active proteins and which are at least 80% identical to the nucleic acid sequences of reported herein. More preferred n21 nucleic acid fragments are at least 90% identical to the sequences herein. Most preferred are n21 nucleic acid fragments that are at least 95% identical to the nucleic acid fragments reported herein.

Comparison of the l14 nucleotide base and deduced amino acid sequences to public databases reveals that the most similar known sequences range from about 51 % identical to the amino acid sequence of l14 reported herein over length of 454 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred l14 encoding nucleic acid sequences corresponding to the instant ORF's are those encoding active proteins and which are at least 80% identical to the nucleic acid sequences of reported herein. More preferred l14 nucleic acid fragments are at least 90% identical to the sequences herein. Most preferred are l14 nucleic acid fragments that are at least 95% identical to the nucleic acid fragments reported herein.

Comparison of the k7 nucleotide base and deduced amino acid sequences to public databases reveals that the most similar known sequences range from about 49% identical to the amino acid sequence of k7 reported herein over length of 459 amino acids using a BLASTP analysis (Altschul et al., J. Mol. Biol 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred k7 encoding nucleic acid sequences corresponding to the instant ORF's are those encoding active proteins and which are at least 80% identical to the nucleic acid sequences of reported herein. More preferred k7 nucleic acid fragments are at least 90% identical to the sequences herein. Most preferred are k7 nucleic acid fragments that are at least 95% identical to the nucleic acid fragments reported herein.

Comparison of the e12 nucleotide base and deduced amino acid sequences to public databases reveals that the most similar known sequences range from about 50% identical to the amino acid sequence of e12 reported herein over length of 459 amino acids using a BLASTP analysis (Altschul et al., J. Mol. Biol. 215:403-410 (1990)). More preferred amino acid fragments are at least about 70%-80% identical to the sequences herein, where about 80%-90% is preferred. Most preferred are nucleic acid fragments that are at least 95% identical to the amino acid fragments reported herein. Similarly, preferred e12 encoding nucleic acid sequences corresponding to the instant ORF's are those encoding active proteins and which are at least 80% identical to the nucleic acid sequences of reported herein. More preferred e12 nucleic acid fragments are at least 90% identical to the sequences herein. Most preferred are e12 nucleic acid fragments that are at least 95% identical to the nucleic acid fragments reported herein.

Methods for Isolation of Homologs

The nucleic acid fragments of the instant invention may be used to isolate genes encoding homologous proteins from the same or other microbial species. Isolation of homologous genes using sequence-dependent protocols is well known in the art. Examples of sequence-dependent protocols include, but are not limited to, methods of nucleic acid hybridization and methods of DNA and RNA amplification, as exemplified by various uses of nucleic acid amplification technologies [e.g., polymerase chain reaction (PCR) (Mullis et al., U.S. Pat. No. 4,683,202); ligase chain reaction (LCR) (Tabor et al., Proc. Acad. Sci. USA 82:1074 (1985)); or strand displacement amplification (SDA) (Walker et al., Proc. Natl. Acad. Sci. U.S.A., 89:392 (1992))].

For example, genes encoding similar proteins or polypetides to the present γ-methyleneglutamate decarboxylase, \tilde{α}methylene-γ-aminobutyrate aminotransferase, α-methylene-γ-hydroxybutyrate dehydrogenase, and α-methylene-γ-hydroxybutyrate/UDP-glucose glucosyltransferase could be isolated directly by using all or a portion of the nucleic acid fragments set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23 as DNA hybridization probes to screen libraries from any desired bacteria using methodology well known to those skilled in the art. Specific oligonucleotide probes based upon the instant nucleic acid sequences can be designed and synthesized by methods known in the art (Maniatis). Moreover, the entire sequences can be used directly to synthesize 1.) DNA probes, by methods known to the skilled artisan such as random primers DNA labeling, nick translation, or end-labeling techniques; or 2.) RNA probes using available in vitro transcription systems. In addition, specific primers can be designed and used to amplify a part of or the full-length of the instant sequences. The resulting amplification products can be labeled directly during amplification reactions or labeled after amplification reactions, and used as probes to isolate full length DNA fragments under conditions of appropriate stringency.

Typically, in PCR-type primer directed amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The Use of Oligonucleotide as Specific Hybridization Probes in the Diagnosis of Genetic Disorders”, In, Human Genetic Diseases: A Practical Approach, K. E. Davis, Ed., (1986) pp. 33-50 IRL Press, Herndon, Va.; Rychlik, W. In, Methods in Molecular Biology, B. A. White, Ed., (1993) Vol. 15, pp. 31-39; PCR Protocols: Current Methods and Applications, Humania Press, Inc., Totowa, N.J.).

Generally, PCR primers may be used to amplify longer nucleic acid fragments encoding homologous genes from DNA or RNA. However, the polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes. Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

Accordingly, the instant invention provides a method for identifying a nucleic acid molecule encoding a γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase comprising: (a) synthesizing at least one oligonucleotide primer corresponding to a portion of the sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23; and (b) amplifying an insert present in a cloning vector using the oligonucleotide primer of step (a), wherein the amplified insert encodes a nucleic acid sequence selected from the group consisting of an γ-methyleneglutamate decarboxylase, α-methylene-γ-aminobutyrate aminotransferase, α-methylene-γ-hydroxybutyrate dehydrogenase and an α-methylene-γ-hydroxybutyrate/UDP-glucose glucosyltransferase.

Alternatively, the instant sequences may be employed as hybridization reagents for the identification of homologs. The basic components of a nucleic acid hybridization test include a probe, a sample suspected of containing the gene or gene fragment of interest, and a specific hybridization method. Probes of the present invention are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia, Inc.) (about 300-500 kilodaltons), polyvinylpyrrolidone (about 250-500 kdal) and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt/vol glycine. Other additives may also be included, such as volume exclusion agents that include a variety of polar water-soluble or swellable agents, such as polyethylene glycol, anionic polymers such as polyacrylate or polymethylacrylate, and anionic saccharidic polymers, such as dextran sulfate.

Thus, the instant invention provides a method for identifying a nucleic acid molecule encoding a γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase comprising: (a) probing a genomic library with a portion of a nucleic acid molcule selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23; (b) identifying a DNA clone that hybridizes under conditions of 0.1× SSC, 0.1% SDS, 65° C. and washed with 2× SSC, 0.1% SDS followed by 0.1× SSC, 0.1% SDS with the nucleic acid molecule of (a); and (c) sequencing the genomic fragment that comprises the clone identified in step (b), wherein the sequenced genomic fragment encodes an enzyme from the group consisting of γ-methyleneglutamate decarboxylase, α-methylene-γ-aminobutyrate aminotransferase, α-methylene-γ-hydroxybutyrate dehydrogenase and α-methylen\tilde{e}γ-hydroxybutyrate/UDP-glucose glucosyltransferase.

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized a nucleic acid probe that is unlabeled and complementary to one portion of the sequence.

Availability of the instant nucleotide and deduced amino acid sequences facilitates immunological screening of DNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen DNA expression libraries to isolate full-length DNA clones of interest (Lerner, R. A. Adv. Immunol. 36:1 (1984); Maniatis).

Microbial Recombinant Expression

The genes and gene products of the instant γ-methyleneglutamate decarboxylase, α-methylene-γ-aminobutyrate aminotransferase, α-methylene-γ- hydroxybutyrate dehydrogenase and α-methylene-γ-hydroxybutyrate/UDP-glucose glucosyltransferase sequences may be introduced into heterologous host cells, particularly in the cells of microbial hosts.

Host cells preferred for expression of the instant genes and nucleic acid molecules are microbial hosts that can be found broadly within the fungal or bacterial families and which grow over a wide range of temperature, pH values, and solvent tolerances. For example, it is contemplated that any bacteria, yeast, algae and filamentous fungi will be suitable hosts for expression of the present nucleic acid fragments. Because transcription, translation, and the protein biosynthetic apparatus is the same irrespective of the cellular feedstock, functional genes are expressed irrespective of carbon feedstock used to generate cellular biomass. Large scale microbial growth and functional gene expression may utilize a wide range of simple or complex carbohydrates, organic acids and alcohols, and saturated hydrocarbons such as methane or carbon dioxide in the case of photosynthetic or chemoautotrophic hosts. However, the functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression.

Examples of suitable host strains include, but are not limited to: fungal or yeast species such as Aspergillus, Trichoderma, Saccharomyces, Pichia, Candida and Hansenula; or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium, Klebsiella, Burkholderia, Sphingomonas, Brevibacterium, Corynebacterium, Mycobacterium, Arthrobacter, Nocardia, Actinomyces and Comamonas.

Microbial expression systems and expression vectors containing regulatory sequences that direct high level expression of foreign proteins are well known to those skilled in the art. Any of these could be used to construct chimeric genes for production of the any of the gene products of the instant sequences. These chimeric genes could then be introduced into appropriate microorganisms via transformation to provide high level expression of the enzymes.

Accordingly, it is expected for example that introduction of chimeric genes encoding the instant plant enzymes under the control of the appropriate promoters will demonstrate increased production of tuliposide A and its pathway intermediates. It is contemplated that it will be useful to express the instant genes both in natural host cells as well as heterologous hosts. Introduction of the present genes into the native host will result in elevated levels of existing production of tuliposide A. Additionally, the instant genes may also be introduced into non-native host bacteria where there are advantages to manipulate the tuliposide A production that are not present in the organisms from which the instant genes are directly isolated.

Vectors

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Promoters and Termination Control Regions

Initiation control regions or promoters, which are useful to drive expression of the instant ORF's in the desired host cell, are numerous and familiar to those skilled in the art. Virtually any promoter capable of driving these genes is suitable for the present invention including but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, and npr promoters and various phage promoters useful for expression in Bacillus.

Termination control regions may also be derived from various genes native to the preferred hosts. Optionally, a termination site may be unnecessary; however, it is most preferred if included.

Plant Host Systems

The instant invention can also be used to transform a suitable plant host for the production of tulipalin A, tuliposide A, or any tuliposide A pathway intermediates. Virtually any plant host that is capable of supporting the expression of a tuliposide A synthesizing gene will be suitable, however crop plants are preferred for their ease of harvesting and large biomass. Suitable plant hosts will include but are not limited to both monocots and dicots such as soybean, rapeseed (Brassica napus, B. campestris), sunflower (Helianthus annus), cotton (Gossypium hirsutum), corn, tobacco (Nicotiana tabacum), alfalfa (Medicago sativa), wheat (Triticum sp.), barley (Hordeum vulgare), oats (Avena sativa, L), sorghum (Sorghum bicolor), rice (Oryza sativa), Arabidopsis, cruciferous vegetables, melons, carrots, celery, parsley, tomatoes, potatoes, strawberries, peanuts, grapes, grass seed crops, sugar beet, sugar cane, canola, millet, beans, peas, rye, flax, hardwood trees, softwood trees, and forage grasses.

Overexpression of the tuliposide A synthesizing proteins may be accomplished by first constructing chimeric genes of the present invention in which the coding regions are operably linked to promoters capable of directing expression of a gene in the desired tissues at the desired stage of development. For reasons of convenience, the chimeric genes may comprise promoter sequences and translation leader sequences derived from the same genes. 3′ Non-coding sequences encoding transcription termination signals must also be provided. The instant chimeric genes may also comprise one or more introns in order to facilitate gene expression.

Any combination of any promoter and any terminator capable of inducing expression of a coding region may be used in the chimeric genetic sequence. Some suitable examples of promoters and terminators include those from nopaline synthase (nos), octopine synthase (ocs) and cauliflower mosaic virus (CaMV) genes. One type of efficient plant promoter that may be used is a high level plant promoter. Such promoters, in operable linkage with the genetic sequences of the present invention should be capable of promoting expression of the present gene product. High level plant promoters that may be used in this invention, for example, include the promoter of the small subunit (ss) of the ribulose-1,5-bisphosphate carboxylase from soybean (Berry-Lowe et al., J. Molecular and App. Gen., 1:483-498 (1982)), and the promoter of the chlorophyll a/b binding protein. These two promoters are known to be light-induced in plant cells (see, for example, Genetic Engineering of Plants, an Agricultural Perspective, A. Cashmore, Plenum, N.Y. (1983); pp 29-38; Coruzzi, G. et al., J. Biol. Chem., 258:1399 (1983), and Dunsmuir, P. et al., J. Mol. Appl. Genetics, 2:285 (1983)).

Plasmid vectors comprising the instant chimeric genes can then be constructed. The choice of plasmid vector depends upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus multiple events must be screened in order to obtain lines displaying the desired expression level and pattern. Such screening may be accomplished by Southern analysis of DNA blots (Southern, J. Mol. Biol. 98:503 (1975)), Northern analysis of mRNA expression (Kroczek, J. Chromatogr. Biomed. Appl., 618 (1-2):133-145 (1993)), Western analysis of protein expression, or phenotypic analysis.

For some applications it will be useful to direct the instant proteins to different cellular compartments. It is thus envisioned that the chimeric genes described above may be further supplemented by altering the coding sequences to encode enzymes with appropriate intracellular targeting sequences such as transit sequences (Keegstra, K., Cell 56:247-253 (1989)), signal sequences or sequences encoding endoplasmic reticulum localization (Chrispeels, J. J., Ann. Rev. Plant Phys. Plant Mol. Biol. 42:21-53 (1991)), or nuclear localization signals (Raikhel, N. Plant Phys.100:1627-1632 (1992)) added and/or with targeting sequences that are already removed. While the references cited give examples of each of these, the list is not exhaustive and more targeting signals of utility may be discovered in the future that are useful in the invention.

A variety of techniques are available and known to those skilled in the art for introduction of constructs into a plant cell host. These techniques include transformation with DNA employing A. tumefaciens or A. rhizogenes as the transforming agent, electroporation, particle acceleration, etc. (See for example, EP 295959 and EP 138341). One suitable method involves the use of binary type vectors of Ti and Ri plasmids of Agrobacterium spp. Ti-derived vectors transform a wide variety of higher plants, including monocotyledonous and dicotyledonous plants such as soybean, cotton, rape, tobacco, and rice (Pacciotti et al., Bio/Technology 3:241 (1985); Byrne et al., Plant Cell, Tissue and Organ Culture 8:3 (1987); Sukhapinda et al., Plant Mol. Biol. 8:209-216 (1987); Lorz et al., Mol. Gen. Genet. 199:178 (1985); Potrykus, Mol. Gen. Genet. 199:183 (1985); Park et al., J. Plant Biol. 38(4):365-71 (1995); and Hiei et al., Plant J. 6:271-282 (1994)). The use of T-DNA to transform plant cells has received extensive study and is amply described (EP 120516; Hoekema, In: The Binary Plant Vector System, Offset-drukkerij Kanters B. V.; Alblasserdam (1985), Chapter V; Knauf et al., Genetic Analysis of Host Range Expression by Agrobacterium In: Molecular Genetics of the Bacteria-Plant Interaction, Puhler, A. Ed., Springer-Verlag, New York, 1983, p. 245; and An et al., EMBO J. 4:277-284 (1985)). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (see Fromm et al., Nature (London) 319:791 (1986)) or high-velocity biolistic bombardment with metal particles coated with the nucleic acid constructs (see Kline et al., Nature (London) 327:70 (1987); and U.S. Pat. No. 4,945,050). Once transformed, the cells can be regenerated by those skilled in the art. Of particular relevance are the recently described methods to transform foreign genes into commercially important crops, such as rapeseed (De Block et al., Plant Physiol. 91:694-701 (1989)), sunflower (Everett et al., Bio/Technology 5:1201 (1987)), soybean (McCabe et al., Bio/Technology 6:923 (1988); Hinchee et al., Bio/Technology 6:915 (1988); Chee et al., Plant Physiol. 91:1212-1218 (1989); Christou et al., Proc. Natl. Acad. Sci USA 86:7500-7504 (1989); EP 301749), rice (Hiei et al., Plant J. 6:271-282 (1994)), and corn (Gordon-Kamm et al., Plant Cell 2:603-618 (1990); Fromm et al., Biotechnology 8:833-839 (1990)).

Transgenic plant cells are then placed in an appropriate selective medium for selection of transgenic cells which are then grown to callus. Shoots are grown from callus and plantlets are generated from the shoot by growing in rooting medium. The various constructs normally will be joined to a marker for selection in plant cells. Conveniently, the marker may be resistance to a biocide (particularly an antibiotic such as kanamycin, G418, bleomycin, hygromycin, chloramphenicol, herbicide, or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. Heterologous constructs will contain at least one region that is not native to the gene from which the transcription-initiation-region is derived. To confirm the presence of the transgenes in transgenic cells and plants, a Southern blot analysis can be performed using methods known to those skilled in the art.

Pathway Engineering

Knowledge of the sequence of the present genes will be useful in manipulating the tulipalin A biosynthetic pathway in any organism having such a pathway (or in any organism in which such a pathway is introduced). Methods of manipulating genetic pathways are common and well known in the art. Selected genes in a particularly pathway may be up-regulated or down-regulated by variety of methods. Additionally, competing pathways in an organism may be eliminated or sublimated by gene disruption and similar techniques.

Up-Regulation of Tuliposide A Biosynthesizing Proteins

Once a key genetic pathway has been identified and sequenced, specific genes may be up-regulated to increase the output of the pathway. For example, additional copies of the targeted tuliposide A biosynthesizing gene(s) may be introduced into the host cell on multicopy plasmids such as pBR322. Such genes may also be integrated into the chromosome with appropriate regulatory sequences that result in increased levels. This would be useful when the goal is to increase production of tuliposide A or any of its pathway intermediates. Alternatively the target genes may be modified so as to be under the control of non-native promoters. Where it is desired that a pathway operate at a particular point in a cell cycle or during a fermentation run, regulated or inducible promoters may be used to replace the native promoter of the target gene(s). Similarly, in some cases the native or endogenous promoter may be modified to increase gene expression. For example, endogenous promoters can be altered in vivo by mutation, deletion, and/or substitution (see, Kmiec, U.S. Pat. No. 5,565,350; Zarling et al., PCT/US93/03868). In plant systems, endogenous promoters can be similarly upregulated by insertion of promoter enhancer elements in proximity to endogenous promoters.

Within the context of the present invention, it may be useful to modulate the expression of the identified tuliposide A biosynthetic pathway by any one of the methods described above. For example, the present invention provides several genes encoding key enzymes responsible for the conversion of γ-methyleneglutamate to tuliposide A. The isolated genes include γ-methyleneglutamate decarboxylase, α-methylene-γ-aminobutyrate aminotransferase, α-methylene-γ-hydroxybutyrate dehydrogenase and α-methylene-γ-hydroxybutyrate/UDP-glucose glucosyltransferase genes. In particular, it may be useful to up-regulate the flux of pyruvate and aspartate, thereby increasing the quantities of γ-methyleneglutamate substrate available for conversion to tuliposide A.

Down-Regulation of Tuliposide A Biosynthesizing Proteins

Alternatively, it may be necessary to reduce or eliminate the expression of certain genes in the tuliposide A biosynthesizing pathway (e.g., to produce a tulip with reduced allergic capabilities) or in competing pathways that may serve as competing sinks for energy or carbon. Methods of down-regulating genes for this purpose have been explored.

For example, where sequence of the gene to be disrupted is known, one of the most effective methods for gene down regulation is targeted gene disruption where foreign DNA is inserted into a structural gene so as to disrupt transcription. This is typically referred to as generating a gene “knockout”, defined as the partial or complete suppression of the expression of at least a portion of a protein encoded by an endogenous DNA sequence in a cell. This can be effected by the creation of genetic cassettes (or “knockout constructs”), referring to a nucleic acid sequence that is designed to decrease or suppress expression of a protein encoded by endogenous DNA sequences in a cell. The nucleic acid sequence used as the knockout construct is typically comprised of: (1) DNA from some portion of the gene (exon sequence, intron sequence, and/or promoter sequence) to be suppressed; and (2) a marker sequence used to detect the presence of the knockout construct in the cell. The knockout construct is inserted into a host cell, and integrates with the genomic DNA of the cell in such a position so as to prevent, or interrupt, transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). Thus, introduction of the knockout construct into the host cell results in insertion of the foreign DNA into the structural gene via the native DNA replication mechanisms of the cell (see, for example, Hamilton et al., J. Bacteriol. 171:4617-4622 (1989); Balbas et al., Gene 136:211-213 (1993); Gueldener et al., Nucleic Acids Res. 24:2519-2524 (1996); and Smith et al., Methods Mol. Cell. Biol. 5:270-277(1996)).

Alternative methods are available to reduce or eliminate expression of genes encoding the instant polypeptides, if desirable in plants for some applications. In order to accomplish this, a chimeric gene designed for co-suppression of the instant polypeptide can be constructed by linking a gene or gene fragment encoding that polypeptide to plant promoter sequences. Antisense technology requires that a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA that encodes the protein of interest. A person skilled in the art will know that special considerations are associated with the use of antisense technologies in order to reduce expression of particular genes. For example, the proper level of expression of antisense genes may require the use of different chimeric genes utilizing different regulatory elements known to the skilled artisan. Nonetheless, either the co-suppression or antisense chimeric genes could be introduced into plants via transformation wherein expression of the corresponding endogenous genes is reduced or eliminated.

Finally, one recent variation upon “classical” antisense and cosuppression methodologies is embodied in WO 02/00904, published on Jan. 3, 2002. Specifically, it was found that suitable nucleic acid sequences and their reverse complement can be used to alter the expression of any mRNA encoding a protein of interest which is in proximity to the suitable nucleic acid sequence and its reverse complement. Surprisingly, the suitable nucleic acid sequence and its reverse complement can be either unrelated to any endogenous RNA in the host or can be encoded by any nucleic acid sequence in the genome of the host provided that the nucleic acid sequence does not encode any target mRNA or any sequence that is substantially similar to the target mRNA. A preferred artificial, non-naturally occurring, sequence is that encoded by the peptide “ELVISLIVES” (SEQ ID NO: 67). This approach permits a very efficient and robust approach to achieving single, or multiple, gene co-suppression using single plasmid transformation.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression or similar methodologies thereto (U.S. Pat. No. 5,190,931; U.S. Pat. No. 5,107,065; U.S. Pat. No. 5,283,323; WO 02/00904). An antisense or cosuppression construct would act as a dominant negative regulator of gene activity. While conventional mutations can yield negative regulation of gene activity, these effects are most likely recessive. The dominant negative regulation available with a transgenic approach may be advantageous from a breeding perspective. In addition, the ability to restrict the expression of specific phenotype to the reproductive tissues of the plant by the use of tissue specific promoters may confer agronomic advantages relative to conventional mutations that may have an effect in all tissues in which a mutant gene is ordinarily expressed.

A person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds, and is not an inherent part of the invention. For example, one can screen by looking for changes in gene expression by using antibodies specific for the protein encoded by the gene being suppressed, or one could establish assays that specifically measure enzyme activity. A preferred method will be one that allows large numbers of samples to be processed rapidly, since it will be expected that a large number of transformants will be negative for the desired phenotype.

Although targeted gene disruption and antisense technology offer effective means of down-regulating genes where the sequence is known, other less specific methodologies have been developed that are not sequence based. For example, cells may be exposed to UV radiation and then screened for the desired phenotype. Mutagenesis with chemical agents is also effective for generating mutants and commonly used substances include chemicals that affect nonreplicating DNA such as HNO₂ and NH₂OH, as well as agents that affect replicating DNA such as acridine dyes, notable for causing frameshift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36: 227 (1992). Similar mutagenic techniques using irradiation or chemical methods exist for plants and plant seeds.

Another non-specific method of gene disruption is the use of transposable elements or transposons. Transposons are genetic elements that insert randomly in DNA but can be later retrieved on the basis of sequence to determine where the insertion has occurred. Both in vivo and in vitro transposition methods are known. Both methods involve the use of a transposable element in combination with a transposase enzyme. When the transposable element or transposon is contacted with a nucleic acid fragment in the presence of the transposase, the transposable element will randomly insert into the nucleic acid fragment. The technique is useful for random mutagenesis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass., based upon the bacterial transposon Tn7; and the EZ::TN Transposon Insertion Systems, available from Epicentre Technologies, Madison, Wis., based upon the Tn5 bacterial transposable element).

Plants can also be mutated using similar insertional mutagenic techniques. Various transposon-based techniques (Ac, Mu) are commonly used in plants to randomly insert transposon sequences into the plant genome and thus disrupt gene function. Often plants containing an effective transposase are crossed with plants containing a transposable element, thus initiating random insertions of the transposon throughout the plant genome. In addition, DNA insertions using the Agrobacterium Ti plasmid are commonly used to disrupt plant genes. DNA bordered by the Ti insertional elements in an appropriate shuttle vector are incorporated into the plant genome via an Agrobacterium-mediated process. Other similar systems using Rhizobium are also commonly used. Finally, methods for randomly inserting foreign DNA into plant genomes commonly used by those skilled in the art include, but are not limited to, direct uptake of foreign DNA constructs, electroporation or high-velocity biolistic bombardment with metal particles coated with the nucleic acid constructs.

Research Applications

The instant polypeptides (or portions thereof) may be produced in heterologous host cells, particularly in the cells of microbial hosts, and can be used to prepare antibodies to these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded branched-chain amino acid degradation enzymes. An example of a vector for high level expression of the instant polypeptides in a bacterial host is provided (Example 8).

Additionally, the instant polypeptides can be used as targets to facilitate design and/or identification of inhibitors of those enzymes that may be useful as herbicides. This is desirable because the polypeptides described herein catalyze various steps in degradation of branched-chain amino acids. Accordingly, inhibition of the activity of one or more of the enzymes described herein could lead to inhibition of plant growth. Thus, the instant polypeptides could be appropriate for new herbicide discovery and design.

All or a substantial portion of the nucleic acid fragments of the instant invention may also be used as probes for genetically and physically mapping the genes that they are a part of, and as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al., Genomics 1:174-181 (1987)) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al., Am. J. Hum. Genet. 32:314-331 (1980)).

The production and use of plant gene-derived probes for use in genetic mapping is described in Bernatzky and Tanksley, Plant Mol. Biol. Reporter 4(1):37-41 (1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al., In: Nonmammalian Genomic Analysis: A Practical Guide, Academic Press (1996), pp. 319-346, and references cited therein).

In another embodiment, nucleic acid probes derived from the instant nucleic acid sequences may be used in direct fluorescence in situ hybridization (FISH) mapping (Trask Trends Genet. 7:149-154 (1991)). Although current methods of FISH mapping favor use of large clones (see Laan et al., Genome Research 5:13-20 (1995)), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian, J. Lab. Clin. Med. 114(2):95-96 (1989)), polymorphism of PCR-amplified fragments (CAPS; Sheffield et al., Genomics 16:325-332 (1993)), allele-specific ligation (Landegren et al., Science 241:1077-1080 (1988)), nucleotide extension reactions (Sokolov, Nucleic Acid Res. 18:3671 (1990)), Radiation Hybrid Mapping (Walter et al., Nature Genetics 7:22-28 (1997)) and Happy Mapping (Dear and Cook, Nucleic Acid Res. 17:6795-6807 (1989)). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer, Proc. Natl Acad. Sci USA 86:9402 (1989); Koes et al., Proc. Natl. Acad. Sci USA 92:8149 (1995); Bensen et al., Plant Cell 7:75 (1995)). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptides. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptides can be identified and obtained. This mutant plant can then be used to determine or confirm the natural function of the instant polypeptides disclosed herein.

Protein Engineering

It is contemplated that the present nucleotides may be used to produce gene products having enhanced or altered activity. Various methods are known for mutating a native gene sequence to produce a gene product with altered or enhanced activity including but not limited to error prone PCR (Melnikov et al., Nucleic Acids Research, (Feb. 15, 1999) 27(4): 1056-1062); site directed mutagenesis (Coombs et al., Proteins (1998), 259-311, 1 plate. Editor(s): Angelefti, Ruth Hogue. Publisher: Academic, San Diego, Calif.) and “gene shuffling” (U.S. Pat. No. 5,605,793; U.S. Pat. No. 5,811,238; U.S. Pat. No. 5,830,721; and U.S. Pat. No. 5,837,458, incorporated herein by reference).

The method of gene shuffling is particularly attractive due to its facile implementation and high rate of mutagenesis and ease of screening. The process of gene shuffling involves the restriction endonuclease cleavage of a gene of interest into fragments of specific size in the presence of additional populations of DNA regions of both similarity to or difference to the gene of interest. This pool of fragments will then be denatured and reannealed to create a mutated gene. The mutated gene is then screened for altered activity.

The instant microbial sequences of the present invention may be mutated and screened for altered or enhanced activity by this method. The sequences should be double stranded and can be of various lengths ranging from 50 bp to 10 kb. The sequences may be randomly digested into fragments ranging from about 10 bp to 1000 bp, using restriction endonucleases well known in the art (Maniatis supra). In addition to the instant microbial sequences, populations of fragments that are hybridizable to all or portions of the microbial sequence may be added. Similarly, a population of fragments which are not hybridizable to the instant sequence may also be added. Typically these additional fragment populations are added in about a 10 to 20 fold excess by weight as compared to the total nucleic acid. This process will produce from about 100 to about 1000 different specific nucleic acid fragments in the mixture. The mixed population of random nucleic acid fragments are denatured to form single-stranded nucleic acid fragments and then reannealed. Only those single-stranded nucleic acid fragments having regions of homology with other single-stranded nucleic acid fragments will reanneal. The random nucleic acid fragments may be denatured by heating. One skilled in the art could determine the conditions necessary to completely denature the double stranded nucleic acid. Preferably the temperature ranges from 80° C. to 100° C. The nucleic acid fragments may be reannealed by cooling. Preferably the temperature ranges from 20° C. to 75° C. Renaturation can be accelerated by the addition of polyethylene glycol (“PEG”) or salt. A suitable salt concentration may range from 0 mM to 200 mM. The annealed nucleic acid fragments are then incubated in the presence of a nucleic acid polymerase and dNTP's (i.e., dATP, dCTP, dGTP and dTTP). The nucleic acid polymerase may be the Klenow fragment, the Taq polymerase or any other DNA polymerase known in the art. The polymerase may be added to the random nucleic acid fragments prior to annealing, simultaneously with annealing or after annealing. The cycle of denaturation, renaturation and incubation in the presence of polymerase is repeated for a desired number of times. Preferably the cycle is repeated from 2 to 50 times, more preferably the sequence is repeated from 10 to 40 times. The resulting nucleic acid is a larger double-stranded polynucleotide ranging from about 50 bp to about 100 kb and may be screened for expression and altered activity by standard cloning and expression protocols (Maniatis, supra).

Furthermore, a hybrid protein can be assembled by fusion of functional domains using the gene shuffling (exon shuffling) method (Nixon et al., PNAS, 94:1069-1073 (1997)). The functional domain of the instant gene can be combined with the functional domain of other genes to create novel enzymes with desired catalytic function. A hybrid enzyme may be constructed using PCR overlap extension methods and cloned into various expression vectors using the techniques well known to those skilled in art.

Industrial Production

Where commercial production of tuliposide A pathway intermediates, tuliposide A, or tulipalin A is desired, a variety of culture methodologies may be applied. For example, large-scale production from a recombinant microbial host may be produced by both batch and continuous culture methodologies.

A classical batch culturing method is a closed system where the composition of the media is set at the beginning of the culture and not subjected to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Commercial production of tuliposide A pathway intermediates, tuliposide A, or tulipalin A may also be accomplished with a continuous culture. Continuous cultures are open systems where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

As is well known to those skilled in the art, whole microbial cells can be used as catalyst without any pretreatment such as permeabilization. Alternatively, the whole cells may be permeabilized by methods familiar to those skilled in the art (e.g., treatment with toluene, detergents, or freeze thawing) to improve the rate of diffusion of materials into and out of the cells.

In one embodiment of the invention, it is preferred that the enzyme catalyst be immobilized in a polymer matrix (e.g., alginate, carrageenan, polyvinyl alcohol, or polyacrylamide gel (PAG)) or on a soluble or insoluble support (e.g., celite) to facilitate recovery and reuse of the catalyst. Methods for the immobilization of cells in a polymer matrix or on a soluble or insoluble support have been widely reported and are well known to those skilled in the art.

In addition to production of tuliposide A pathway intermediates, tuliposide A, or tulipalin A in vivo (e.g., within plant or microbial cells), the present invention also encompasses means to produce these compoundss in vitro. For example, any of the tuliposide A synthesizing enzymes can also be isolated from the whole cells and used directly as catalyst, or they can be immobilized in a polymer matrix or on a soluble or insoluble catalyst support. These methods have also been widely reported and are well known to those skilled in the art (Methods in Biotechnology, Vol. 1: Immobilization of Enzymes and Cells; Gordon F. Bickerstaff, Editor; Humana Press, Totowa, N.J.; 1997).

Conversion of Tulipalin A and α-methylene-γ-hydroxybutyrate to Tulipalin A

Alpha-methylene-γ-hydroxybutyrate can be further converted to tulipalin A using any strong acid catalyst generally known and widely available to one skilled in the art, such as, sulfuric acid, hydrochloric acid, nitric acid, glacial acetic acid, or any other soluble organic or inorganic catalyst. The conversion can be performed at standard temperature and pressure conditions.

Tuliposide A, the last product biosynthetically produced in the present invention via the activity of the α-methylene-γ-hydroxybutyrate/UDP-glucose glucosyltransferase enzyme on its α-methylene-γ-hydroxybutyrate substrate, is readily converted to tulipalin A (as shown in FIG. 1) via removal of a glucose molecule. This reaction, whereby glucose is removed from tuliposide A and tulipalin A is formed, may occur by a variety of chemical processes, such as acidic or basic reaction conditions. In addition to chemical processes, the glucose can be removed and tulipalin A formed by an enzyme-catalyzed process. Enzyme-catalyzed processes are often run at ambient temperature, do not require the use of strongly acidic or basic reaction conditions, and do not produce large amounts of unwanted byproducts.

### EXAMPLES

The instant invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Techniques suitable for use in the following Examples may be found in Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)); or by Thomas D. Brock in Biotechnology: A Textbook of Industrial Microbiology, Second Edition, Sinauer Associates, Inc., Sunderland, Mass. (1989). All reagents and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.) or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used the gap creation default value of 12, and the gap extension default value of 4 were used. Where the CGC “Gap” or “Bestfit” programs were used the default gap creation penalty of 50 and the default gap extension penalty of 3 were used. In cases where GCG program parameters were not prompted for, or any other GCG programs, default values were used.

The meaning of abbreviations is as follows: “h” means hour(s), “min” means minute(s), “sec” means second(s), “d” means day(s), μL“means microliter(s), “mL” means milliliter(s), “L” means liter(s), “μm” means micrometer(s), “ppm” means parts per million (i.e., milligrams per liter), and “psi” means pounds per square inch.

### Example 1

### Composition of cDNA Libraries, Isolation and Sequencing of cDNA Clones

cDNA libraries representing mRNAs from Arabidopsis, Alstroemeria, and tulip tissues were prepared. The characteristics of the libraries are described in Table 2.

TABLE 2
cDNA Libraries from Plants

| Library | Species and Tissue |
|---|---|
| ads1n | Arabidopsis (Wassilewskija) - six day old seedling normalized |
| eae1c | Alstroemeria caryophylla - emerging leaf from mature stem |
| eae1s | Alstroemeria caryophylla - emerging leaf from mature stem |
| eae2s | Alstroemeria caryophylla - emerging leaf from mature stem |
| | subtracted with chlorophyll A/B binding protein |
| eal1c | Alstroemeria caryophylla - mature leaf from mature stem |
| etb1c | Tulipa fosteriana (Yellow Emperor) - developing bulbs (10 day |
| | post petal drop) |
| etp1c | Tulipa gesneriana (Apeldoorn) - stage 3 pistil |
| etl1c | Tulipa gesneriana (Apeldoorn) - 3-4 inch emerging leaf |

cDNA libraries may be prepared by any one of several methods available. For example, the cDNAs were introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries were converted into plasmid libraries according to the protocol provided by Stratagene. Upon conversion, cDNA inserts are contained in the plasmid vector pBluescript. In addition, the cDNAs were introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts were in plasmid vectors, plasmid DNAs were prepared from randomly picked bacterial colonies containing recombinant pBluescript plasmids, or the insert cDNA sequences were amplified via polymerase chain reaction using primers specific for vector sequences flanking the inserted cDNA sequences. Amplified insert DNAs or plasmid DNAs were sequenced in dye-primer sequencing reactions to generate partial cDNA sequences (expressed sequence tags or “ESTs”; see Adams et al., Science 252:1651 (1991)). The resulting ESTs were analyzed using a Perkin Elmer Model 377 fluorescent sequencer.

### Example 2

### Identification of cDNA Clones as Putative Tulipalin A Pathway Genes

ESTs encoding candidate tulipalin A biosynthetic genes were identified by conducting BLAST (Basic Local Alignment Search Tool; Altschul et al., J. Mol. Biol. 215:403-410 (1993); see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL and DDBJ databases). The cDNA sequences obtained were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. Nature Genetics 3:266-272 (1993)) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins. cDNAs encoding polypeptides similar to tulipalin biosynthetic genes were identified by searching the database using keyword searches (e.g., “glutamate” and/or “decarboxylase”) or using the TBLASTN algorithm provided by the National Center for Biotechnology Information (NCBI) with known homologs to tulipalin A biosynthetic genes such as plant glutamate decarboxylase, γ-aminobutyrate aminotransferase, γ-hydroxybutyrate dehydrogenase and UDP-glucosyl transferase protein sequences. Clones containing putative tulipalin A biosynthetic genes identified by these means are listed in Table 3.

TABLE 3
cDNAs Identified as Putative Tulipalin A Biosynthetic Genes

| Clone | Biosynthetic Gene Type |
|---|---|
| eae1c.pk004.m24 | glutamate decarboxylase |
| eal1c.pk006.j13 | glutamate decarboxylase |
| eae1c.pk002.a20 | glutamate decarboxylase |
| etp1c.pk001.e20 | γ-aminobutyrate aminotransferase |
| eae2s.pk005.c16 | γ-aminobutyrate aminotransferase |
| etp1c.pk005.h21 | γ-hydroxybutyrate dehydrogenase |
| etp1c.pk005.k10 | γ-hydroxybutyrate dehydrogenase |
| ads1n pk003.i20 | γ-hydroxybutyrate dehydrogenase |
| etp1c.pk001.n21 | UDP-glucosyl transferase |
| etp1c.pk005.I14 | UDP-glucosyl transferase |
| eae1c.pk005.k7 | UDP-glucosyl transferase |
| eae1c.pk006.e12 | UDP-glucosyl transferase |

### Example 3

### Cloning and Characterization of γ-Methyleneglutamate Decarboxylases

As described in Examples 1 and 2, three clones containing cDNAs were identified from the Alstroemeria EST libraries that have homology to known glutamate decarboxylases. The cDNA inserts contained in these clones, eae1c.pk004.m24, eal1c.pk006.j13 and eae1c.pk002.a20, were fully sequenced. The complete DNA sequence of the cDNA from clone eal1c.pk006.j13, called gad2 (SEQ ID NO: 1) was obtained using the following primers: M13 forward (−20) (SEQ ID NO: 25), M13 reverse (SEQ ID NO: 26), NW5 (SEQ ID NO: 27), NW6 (SEQ ID NO: 28), and NW7 (SEQ ID NO: 29). The DNA sequence encoded a protein, called GAD2 (SEQ ID NO: 2), that had homology to known plant glutamate decarboxylases. Based on alignments with these known plant glutamate decarboxylases, GAD2 was found to be a full-length cDNA containing a complete ORF. The complete DNA sequence of the cDNA from clone eal1c.pk004.m24, called gad1 (SEQ ID NO: 30), and from clone eal1c.pk006.j13, called gad3(SEQ ID NO: 31) were obtained using the M13 forward (−20) (SEQ ID NO: 25) and M13 reverse (SEQ ID NO: 26) primers. These sequences were partial-length cDNAs lacking the 5′ end of the ORF based on alignments with known plant glutamate decarboxylases. The DNA sequence of gad1 (SEQ ID NO: 30) was identical to a portion of gad2 (SEQ ID NO: 1) and was not further characterized.

Cloning the 5′ End of GAD3:

In order to clone the 5′ end of gad3 (SEQ ID NO: 31), the following primers were designed: NW12 (SEQ ID NO: 32), NW13 (SEQ ID NO: 33), NW14 (SEQ ID NO: 34) and NW15 (SEQ ID NO: 35). The primers were designed close to the existing 5′ end of the partial clone to increase the chance of obtaining a fragment containing the start codon of the gene. Glycerol stocks of the Alstroemeria libraries described in Example 1 (eae1c and eal1c) were obtained, cultures were grown from these stocks and plasmid DNA was isolated using a QIAprep-spin miniprep kit. Two putative 5′ end fragments, of approximately 900 bp and 1,400 bp in size, were amplified from the eal1c library with primer NW14 (SEQ ID NO: 34) and a pBluescript T3 primer (Stratagene; SEQ ID NO: 36). These fragments were subcloned into the pCRII-TOPO vector (Invitrogen) following the manufacturer's protocol and sequenced using M13 forward (−20) (SEQ ID NO: 25) and M13 reverse (SEQ ID NO: 26) primers. The 1,400 bp fragment was additionally sequenced using primers NW18 (SEQ ID NO: 37) and NW19 (SEQ ID NO: 38). A contiguous DNA fragment was assembled from the sequencing results using Sequencher (Gene Codes Corp.). Primers NW20 (SEQ ID NO: 39) and NW21 (SEQ ID NO: 40) were designed and these primers were used to amplify a 1.8 kb fragment from the Alstroemeria libraries. This fragment was subcloned into the pCRII-TOPO vector (Invitrogen) following the manufacturer's protocol affording construct pNW6. pNW6 was sequenced using M13 forward (−20) (SEQ ID NO: 25), M13 reverse (SEQ ID NO: 26), NW18 (SEQ ID NO: 37), NW19 (SEQ ID NO: 38) and NW21 (SEQ ID NO: 40) primers. A comparison of the sequence of the 1.8 kb fragment containing the complete ORF with Gad3 (SEQ ID NO: 31) revealed an in-frame deletion at the 3′ end of the newly sequenced ORF. The 1.8 kb fragment containing the complete ORF was called gad3del (SEQ ID NO: 3). This DNA sequence encoded a protein (SEQ ID NO: 4) with homology to known plant glutamate decarboxylases (see Table 4).

Isolating gad3 Without Inframe Deletion:

Primers NW14 (SEQ ID NO: 34) and NW20 (SEQ ID NO: 39) were used to amplify a DNA fragment from the 1,400 bp-sized PCR product described above. Primers NW22 (SEQ ID NO: 41) and NW23 (SEQ ID NO: 42) were used to amplify a DNA fragment from Gad3 (SEQ ID NO: 31). Products were cleaned using Qiagen gel extraction kit and mixed together in equimolar amounts. Products were denatured at 94° C. and reannealed at 55° C. Using a proofreading polymerase (Elongase, GIBCO), double stranded DNA was synthesized and used as a template in PCR using primers NW20 (SEQ ID NO: 39) and NW22 (SEQ ID NO: 41). The PCR fragment was sub-cloned into the pCRII-TOPO vector giving construct pNW8. pNW8 was sequenced using M13 forward (−20) (SEQ ID NO: 25) and M13 reverse (SEQ ID NO: 26) primers and the new chimeric DNA sequence, called gad3chim (SEQ ID NO: 5), encoded a protein (SEQ ID NO: 6) that had homology to known plant glutamate decarboxylases.

Sequence Analysis:

The ORF of gad2 (SEQ ID NO: 1), gad3del (SEQ ID NO: 3) and gad3chim (SEQ ID NO: 5) were translated into their corresponding protein sequence using Vector NTI Deluxe version 6.2 and each protein sequence was compared to sequences in the “nr” database using BLASTP analysis (Matrix, Blosum62; Gap existence cost, 11; Per residue gap cost, 1; Expect, 10; Descriptions, 50; Alignments, 50; Filter, None). Results of the comparison are shown in Table 4. BLAST results indicated that GAD2, GAD3del and GAD3chim all have the greatest homology to the glutamate decarboxylase isozyme 1 from Nicotiana tabacum.

TABLE 4

| | | SEQ | SEQ | % | % | |
|---|---|---|---|---|---|---|
| EST | | ID | ID | Iden- | Simi- | E- |
| Name | Similarity Identified | base | Peptide | tity^{a} | larity^{b} | value^{c} |
| GAD2 | >gb\|AAC24195.1\| | 1 | 2 | 78 | 87 | 0 |
| | (AF020425) | | | | | |
| | glutamate | | | | | |
| | decarboxylase | | | | | |
| | isozyme 1 | | | | | |
| | [Nicotiana tabacum] | | | | | |
| GAD3 | >gb\|AAC24195.1\| | 3 | 4 | 74 | 85 | 0 |
| del | (AF020425) | | | | | |
| | glutamate | | | | | |
| | decarboxylase | | | | | |
| | isozyme 1 | | | | | |
| | [Nicotiana tabacum] | | | | | |
| GAD3 | >gb\|AAC24195.1\| | 5 | 6 | 74 | 85 | 0 |
| chim | (AF020425) | | | | | |
| | glutamate | | | | | |
| | decarboxylase | | | | | |
| | isozyme 1 | | | | | |
| | [Nicotiana tabacum] | | | | | |

^{a}% Identity is defined as percentage of amino acids that are identical between two proteins.
^{b}% Similarity is defined as percentage of amino acids that are identical or conserved between two proteins.
^{c}Expect value. The Expect value estimates the statistical significance of the match, specifying the number of matches, with a given score, that are expected in a search of a database of this size absolutely by chance.

### Example 4

### Expression Constructs with gad2, gad3del and gad3chim Genes

Primers were designed to the ORFs of gad2 (SEQ ID NO: 1), gad3del (SEQ ID NO: 3) and gad3chim (SEQ ID NO: 5) in order to subclone them into either the pET28a or pET29a expression vectors (Novagen). Various restriction sites were incorporated into the primers to allow for cloning into different restriction sites in each vector.

Vector pNW2 was constructed for expression of the ORF of gad2 (SEQ ID NO: 1). Primers NW9 (SEQ ID NO: 43) and NW10 (SEQ ID NO: 44) were used to amplify the ORF and the resulting PCR product was digested with the restriction enzymes Ncol and HindIII, as was the expression vector pET28a (Novagen). The DNA fragments were purified, ligated and transformed into Escherichia coli DH5α.

Vectors pNW12 and pNW9 were constructed for expression of the ORFs of gad3del (SEQ ID NO: 3) and gad3chim (SEQ ID NO: 5), respectively, as N-terminal His-tag fusions. Primers NW24 (SEQ ID NO: 45) and NW25 (SEQ ID NO: 46) were used to amplify the ORFs and the resulting PCR products were digested with the restriction enzymes Spel and HindIII. The expression vector pET28a (Novagen) was digested with Nhel and HindIII. The digested PCR fragments and vector were purified, ligated and transformed into DH5αcells. All pET expression vector plasmids (pNW2, pNW12 and pNW9) were isolated and purified and the sequence verified by sequencing with the T7 and T7-terminator primers (Novagen).

### Example 5

### Expression of gad2, gad3del and gad3chim Genes

Chemically competent BL21 (DE3) cells (Novagen) were transformed with pNW2, pNW12 and pNW9, as well as with plasmids pET28a and pET29a. An isolated colony of freshly transformed cells was inoculated into 2 mL of LB containing kanamycin (50 μg/mL) and the culture grown overnight at 37° C. Cultures were transferred to 250 mL of LB containing kanamycin (50 μg/mL) and grown at 22° C. to OD 0.6-1.0, at which point they were induced with IPTG (0.1 mM). Induced cells were grown for an additional 14 h and harvested by centrifugation at 10,000× g.

Cells were resuspended in 5 mL of extraction buffer (50 mM Tris-HCl, pH 7.5, 0.2 mM EDTA, 10% glycerol, 1 mM DTT and 1 mM fresh AEBSF) and disrupted by two passes through a French press cell (12,000 psi). Soluble protein extracts were obtained from the supernatant fraction after centrifugation at 10,000× g for 10 min. Protein concentrations were determined using a Bradford reagent (Bio-Rad) (Bradford, Anal. Biochem. 72:248-254 (1976)).

Proteins were separated by SDS-PAGE (10% polyacrylamide) using XCELL II Mini cell (NOVEX) system. By staining the gels and by immunoblot analysis, the estimated molecular weight for Gad2 (SEQ ID NO: 1), Gad3del (SEQ ID NO: 3) and Gad3chim (SEQ ID NO: 5) were about 56 to 58 kD. These protein sizes were similar to the estimated molecular masses derived from the deduced amino acids sequences of their corresponding ORFs. The expressed proteins were more abundant in the insoluble fractions than those in the soluble fractions.

Enzyme activity was determined using a method similar to that of Snedden et al. (J. Biol. Chem. 271:4148-4153 (1996)) and Turano et al. (Plant Physiol. 117:1141-1421 (1998)). Assays were carried out in a volume of 200 μL with 200 mM pyridine-HCl, pH 5.8, 10 mM NaCl, 0.1 mM PLP, 20 mM L-glutamate or D,L-γ-methyleneglutamate, and 20 to 50 uL of enzyme extract. The reactions were incubated at room temperature and 50 μL aliquots were removed at various times. Ethanol (100 μL) was added to terminate the reaction and precipitate the proteins. Samples were centrifuged at 10,000× g for 10 min. The supernatants were transferred to a fresh tube and dried under vacuum after which they were dissolved in 500 μL of borate buffer (400 mM, pH 10). For HPLC analysis, 20 μL of the sample was derivatized by mixing with 20 μL of FMOC ((9-fluorenylmethoxycarbonyl)). FMOC-derivatized samples were separated on a Novapak C18 column (3.9×150 mm, Waters) using an Alliance 2690 HPLC with a 9600 PDA detector (Waters). Samples were injected (16 μL) and separated with a gradient of Buffer A (40 mM NaPO₄, pH 7.8) to Buffer B (methanol:acetonitrile:water/45:45:10). Assay products were FMOC-derivatized and quantified by comparison with standards. In each experiment, enzyme activity determinations were performed in triplicate. Proteins from Escherichia coli expressing empty vectors pET28a and pET29a were used as controls.

Proteins from gad2-expressing cells had enzyme activity against both L-glutamate and D,L-γ-methyleneglutamate. The enzyme activity with L-glutamate was sixteen times higher than with D,L-γ-methyleneglutamate as substrate (see Table 5). Proteins from gad3del- and gad3chim-expressing cells were also active against both L-glutamate and D,L-γ-methyleneglutamate as substrates, but with much higher enzyme activity against D,L-γ-methyleneglutamate than L-glutamate (see Table 5). The higher substrate specificity of the proteins from gad3del (SEQ ID NO: 3) and gad3chim (SEQ ID NO: 5) for D,L-γ-methyleneglutamate suggests that this gene is responsible for biosynthesis of γ-amino-α-methylenebutyrate in Alstroemeria plants. This transformation is one of the key steps in tulipalin A biosynthesis.

TABLE 5

| | Rate for each substrate | | |
|---|---|---|---|
| | (μmol/min⁻¹/mg⁻¹) (crude protein) | | |
| Expression | Glutamate | γ-methyleneglutamate | Ratio |
| vectors | (1) | (2) | (½) |
| control/pET28a | 0 | 0 | N/A |
| Gad2/pNW2 | 12.0 | 0.8 | 16 |
| Gad3del/pNW1 | 7.0 | 12.4 | 0.56 |
| 2 | | | |

### Example 6

### Cloning and Characterization of γ-Aminobutyrate Aminotransferase

Clones from the tulip pistil library (etp1c.pk001.e20) and Alstroemeria leaf library (eae2s.pk005.c16) were identified that had homology to known plant aminotransferases, more specifically δ-, γ-aminotransferases, as described in Examples 1 and 2. Plasmid DNA containing each cDNA was purified and the complete cDNA sequence of the cDNA from clone etp1c.pk001.e20, called e20 (SEQ ID NO: 7) and clone eae2s.pk005.c16, called c16 (SEQ ID NO: 9) was obtained. In addition, the gabT gene from E. coli (SEQ ID NO: 11) was cloned from genomic DNA using information from the NCBI database.

The DNA sequences were translated into their corresponding protein sequences, E20 (SEQ ID NO: 8), C16 (SEQ ID NO: 10) and GABT (SEQ ID NO: 12), respectively, using EditSeq (DNASTAR, Inc.) and each was compared to sequences in the “nr” database using BLASTP analysis (Matrix, Blosum62; Gap existence cost, 11; Per residue gap cost, 1; Expect, 10; Descriptions, 50; Alignments, 50; Filter, None). Results of the comparison are shown in Table 6.

TABLE 6

| | | SEQ | SEQ | % | % | |
|---|---|---|---|---|---|---|
| EST | | ID | ID | Iden- | Simi- | E- |
| Name | Similarity Identified | Base | Peptide | tity^{a} | larity^{a} | value^{c} |
| E20 | gi\|11994739\|dbj\|BAB03068.1 | 7 | 8 | 77 | 86 | 0 |
| | aminotransferase-like protein | | | | | |
| | [Arabidopsis thaliana] | | | | | |
| C16 | gi\|11994739\|dbj\|BAB03068.1 | 9 | 10 | 80 | 89 | 0 |
| | aminotransferase-like protein | | | | | |
| | [Arabidopsis thaliana] | | | | | |
| GABT | gi\|120779\|sp\|P22256 | 11 | 12 | 100 | 100 | 0 |
| | 4-aminobutyrate | | | | | |
| | aminotransferase | | | | | |
| | [Escherichia coli] | | | | | |

^{a}% Identity is defined as percentage of amino acids that are identical between two proteins.
^{b}% Similarity is defined as percentage of amino acids that are identical or conserved between two proteins.
^{c}Expect value. The Expect value estimates the statistical significance of the match, specifying the number of matches, with a given score, that are expected in a search of a database of this size absolutely by chance.

Cloning of Plant Aminotransferase ESTs and E. coli gabT into a Heterologous Expression System:

Primers E20-5 (SEQ ID NO: 47) and E20-3 (SEQ ID NO: 48) were used to amplify e20 (SEQ ID NO: 7). Primers C16-5 (SEQ ID NO: 49) and C16-3 (SEQ ID NO: 50) were used to amplify c16 (SEQ ID NO: 9). Primers gabT-5 (SEQ ID NO: 51) and gabT-3 (SEQ ID NO: 52) were used to amplify the 1281 bp gabT gene of E. coli (SEQ ID NO: 11). PCR products were gel purified (1 % agarose gel in TAE buffer) using the QIAquick Gel Extraction Kit (Qiagen) and eluted with 50 μL of EB buffer (10 mM Tris-HCl, pH 8.0). PCR products were combined with 1 μL of pTrcHis2-TOPO vector (Invitrogen) and incubated at room temperature for 5 min. Two μL of the ligation mix was added to 50 μL of TOP10 chemically competent E. coli cells (Invitrogen). The cells were incubated on ice for 30 min, heated to 42° C. for 30 sec and incubated again on ice for 5 min. SOC media (250 μL) was added to each transformation and the mixture was incubated at 37° C. with shaking at 250 rpm for 1 hour. Subsequently, a series of 10 μL, 25 μL, 50 μL, and 150 μL was plated onto LB media containing ampicillin (100 μg/mL). These plates were incubated overnight at 37° C. Colonies that grew on the selective media were randomly picked and transferred into 250 μL LB liquid media containing ampicillin (100 μg/mL); these colonies were grown for 3 hours at 37° C. with shaking at 250 rpm. Aliquots of these cultures were analyzed by PCR to screen for the insert in these colonies. Positive colonies were grown in 5 mL LB liquid media containing ampicillin (100 μg/mL) overnight at 37° C. with shaking at 250 rpm. Plasmid DNA was isolated using a miniprep kit (Qiagen). Plasmid DNA was subjected to restriction digest analysis to confirm the correct gene orientation in the plasmid. The plasmid containing the correct genes were then transformed into BL21 (DE3) strain E. coli cells. Transformants were plated on LB media containing ampicillin (100 μg/μL) and grown overnight at 37° C. In addition, the pTrcHis2/lacZ plasmid was also transformed into BL21 (DE3) E. coli cells and used as a negative control in expression and enzyme assay experiments.

Expression Studies of Aminotransferases:

Freshly transformed E. coli colonies containing pTrcHis2-TOPO with aminotransferase inserts or pTrcHis2/lacZ were inoculated into 30 mL of LB liquid media containing ampicillin (100 μg/μL) and grown overnight at 37° C. with shaking at 250 rpm. The next morning, the cultures were induced with IPTG at a final concentration of 0.8 mM. The cultures were grown at 37° C. with shaking at 250 rpm for 3 hours. The cultures were then centrifuged at 8,000× g for 10 min and the supernatant was decanted; the resulting pellet was resuspended in 2 mL of buffer (100 mM Phosphate, 1 mM DTT, pH 7.5). Cells were broken by passage through a french press and the resulting extract was centrifuged at 13,000× g for 12 min at 4° C. The supernatant was removed (soluble protein) and the pellet (insoluble protein) was resuspended in 1 mL of SDS-PAGE loading buffer (50 mM Tris-HCl, 100 mM DTT, 2% SDS, 0.1 % bromophenol blue, 10% glycerol, pH 6.8). Approximately 20 μg of total soluble protein and total insoluble protein was loaded onto a 4-12% Bis-Tris Gel (NuPAGE, Invitrogen). The aminotransferase ESTs and lacZ proteins were expressed with a C-terminal polyhistidine tag. Following transfer to a nitrocellulose membrane by Western blotting, expression was confirmed by chemiluminescent detection of the polyhistidine fusion proteins using a India HisProbe (Pierce).

Soluble protein extracts were assayed for aminotransferase activity. Assays contained 1,000 μg of total soluble protein extract, pyridoxal phosphate (1 mM), α-methylene-γ-aminobutyrate (10 mM), and pyruvate (10 mM) for plant enzymes, or α-ketoglutarate (10 mM) for gabT E. coli enzyme, in a total volume of 200 μL. Assays were incubated at 37° C. for 90 min. The enzyme reactions were stopped by freezing to −20° C. Assays were subsequently thawed and (to remove protein) filtered using a 10,000 MWCO microcon filter (Amicon). The amino acid products of the enzymatic reaction were analyzed as their FMOC (9-fluorenylmethoxycarbonyl) derivatives using an LC/MS system. The FMOC derivatives of amino acids in the reaction were prepared by mixing 20 μL of the enzyme reaction, 32 μL of 0.4 N borate buffer, and 50 μL FMOC reagent (11 mM, Agilent Technologies) in a final volume of 200 μL. This mixture was diluted 2 fold and then analyzed using an LC/MS coupled system. Twenty μL of the filtered assay was injected onto a C18 5μ Reverse Phase column (15 cm×2.1 mm; Alltima, Alltech) on a Waters 2690 HPLC coupled to a Micromass Platform LCZ mass spectrometer with parameters optimized using FMOC derivatives of glutamate, alanine and γ-aminobutyrate as standards. The following HPLC method was utilized to separate the reaction components: 0-4.8 min 100% ammonium acetate (1 mM, pH 7.8), 4.8-5 min a linear gradient from 100% ammonium acetate to 17.5% acetonitrile, 5-15 min 17.5% acetonitrile, 15-17 min a linear gradient from 17.5% acetonitrile to 35% acetonitrile, 17-27 min 35% acetonitrile, 27-30 min a linear gradient from 35% acetonitrile to 100% acetonitrile, 30-40 min 100% acetonitrile and 40-42 min a linear gradient from 100% acetonitrile to 100% ammonium acetate. In assays utilizing the gabT protein and the substrates α-methylene-γ-aminobutyriate acid and α-ketoglutarate the expected product is glutamate. In the LC/MS analysis of the assays using gabT, glutamate eluted at 13 min in a solvent of 17.5% acetonitrile and was detected as the FMOC-sodium ion (m/z 392). In assays using etp1c.pk001.e20 protein and the substrates α-methylene-γ-aminobutyrate and pyruvate the product formed was alanine. Alanine eluted at 24 min in a solvent of 35% acetonitrile and was detected as the FMOC-sodium ion (m/z 334). The results showed that gabT (SEQ ID NO: 11) and e20 (SEQ ID NO: 7) are able to transaminate α-methylene-γ-aminobutyrate.

### Example 7

Cloning and Characterization of γ-Hydroxybutyrate Dehydrogenase Sequence Analysis:

As described in Example 2, two clones from the tulip pistil library (etp1c.pk005.k10 and etp1c.pk005.h21) and one clone from Arabidopsis (ads1n.pk003.i20) were identified that had homology to plant γ-hydroxybutyrate dehydrogenase. Based on the similarity of the 5′ ends of each of the EST sequences to known plant γ-hydroxybutyrate dehydrogenases, all three clones were determined to be full-length cDNAs. Plasmid DNA containing each cDNA was purified and the complete cDNA sequence was obtained for clone etp1c.pk005.k10, called Ghbd1 (SEQ ID NO: 13) and ads1n.pk003.i20, called Ghbd2 (SEQ ID NO: 15) using the the M13 forward (−20) (SEQ ID NO: 25), M13 reverse (SEQ ID NO: 26) primers. The two tulip pistil clones were found to be identical and so only etp1c.pk005.k10 was characterized further. The DNA sequences were translated into their corresponding protein sequences, GHBD1 (SEQ ID NO: 14) and GHBD2 (SEQ ID NO: 16) using EditSeq (DNASTAR, Inc.) and each was compared to sequences in the “nr” database using BLASTP analysis (Matrix, Blosum62; Gap existence cost, 11; Per residue gap cost, 1; Expect, 10; Descriptions, 50; Alignments, 50; Filter, None). Results of the comparison are shown in Table 7.

BLAST results show that the protein sequence with the greatest homology to GHBD1 (SEQ ID NO: 14) is a dehydrogenase-like protein from Arabidopsis thaliana. This protein was 81% identical and 90% similar to GHBD1 (SEQ ID NO: 14). Sequence analysis indicated that GHBD2 (SEQ ID NO: 16) was 100% identical to the dehydrogenase-like protein from Arabidopsis thaliana. Both GHBD1 (SEQ ID NO: 14) and GHBD2 (SEQ ID NO: 16) also had significant similarity to 3-hydroxyisobutyrate dehydrogenases which are involved in branch chain amino acid degradation.

TABLE 7

| | | SEQ | SEQ | % | % | |
|---|---|---|---|---|---|---|
| EST | | ID | ID | Iden- | Simi- | E- |
| Name | Similarity Identified | Base | Peptide | tity^{a} | larity^{b} | value^{c} |
| GHBD | dbj\|BAB01322.1\| | 13 | 14 | 81 | 90 | e-130 |
| 1 | (AB025639) | | | | | |
| | dehydrogenase-like | | | | | |
| | protein [Arabidopsis | | | | | |
| | thaliana] | | | | | |
| GHBD | dbj\|BAB01322.1\| | 15 | 16 | 100 | 100 | e-162 |
| 2 | (AB025639) | | | | | |
| | dehydrogenase-like | | | | | |
| | protein [Arabidopsis | | | | | |
| | thaliana] | | | | | |

^{a}% Identity is defined as percentage of amino acids that are identical between two proteins.
^{b}Similarity is defined as percentage of amino acids that are identical or conserved between two proteins.
^{c}Expect value. The Expect value estimates the statistical significance of the match, specifying the number of matches, with a given score, that are expected in a search of a database of this size absolutely by chance.

### Example 8

### Cloning γ-Hydroxybutyrate Dehydrogenase into an Expression Vector

Primers ETP5 (SEQ ID NO: 53) and ETP3 (SEQ ID NO: 54) were used to amplify the 870 bp ORF of ghbd1 (SEQ ID NO: 13). Primers ADS5 (SEQ ID NO: 55) and ADS3 (SEQ ID NO: 56) were used to amplify the 867 bp ORF of ghbd2 (SEQ ID NO: 15). In both cases, PWO DNA polymerase (Boeringer Manhiem) was used in a 50 μL reaction with 1× PWO buffer containing 1.5 mM MgSO₄ (Boeriner Manhiem) and 0.2 mM dNTPs. Standard PCR cycling and temperature conditions were used. After PCR, 0.5 μL of Taq DNA polymerase was added and the reaction was heated at 72° C. for 10 min in order to add an A-tail to the PCR products. PCR products were gel purified (1% agarose gel in TAE buffer) using the Qiagen PCR gel extraction kit (Qiagen) and eluted with 50 μL of EB buffer (10 mM Tris HCl, pH 8.0). PCR products (4 μL) were combined with 1 μL of pTrcHis-TOPO vector and incubated at room temperature for 5 min. After incubation, 2 μL of the ligation mix was mixed with 20 μL of TOP10 chemically competent cells (Invitrogen), incubated on ice for 30 min, heated at 42° C. for 30 sec and incubated again on ice for 5 min. SOC media (250 μL) was added to each transformation, the mixture was incubated at 37° C. for 1 h and 50 μL was plated onto LB media containing ampicillin (100 μg/mL). These plates were incubated overnight at 37° C. Colonies that grew on the selective media were randomly picked and grown in LB liquid media containing ampicillin (100 μg/mL) overnight at 37° C. and 250 rpm. Plasmid DNA was isolated using a Qiagen miniprep kit (Qiagen). DNA was sequenced using the pTrcHis forward (SEQ ID NO: 57) and reverse (SEQ ID NO: 58) primers (Invitrogen) to confirm the correct gene orientation and sequence. The pTrcHis vector containing ghbd1 (SEQ ID NO: 13) was called pTrcEtp and the pTrcHis vector containing ghbd2 (SEQ ID NO: 15) was called pTrcAds.

Expression Studies and Protein Characterization:

Plasmids pTrcEtp and pTrcAds were retransformed into TOP10 cells and freshly plated transformants were used in gene expression studies. In addition, the pTrcHis-TOPO/lacZ plasmid (Invitrogen) was also transformed into TOP10 cells to be used as a negative control in expression experiments. Freshly transformed Escherichia coli colonies containing pTrcEtp, pTrcAds or pTrcHis-TOPO/lacZ were inoculated into 2 mL of LB liquid media containing ampicillin (100 μg/mL) and grown at 37° C. for 5 h. These were then transferred to 100 mL of LB liquid media containing ampicillin (100 μg/mL) and cultures were grown overnight at room temperature. The next morning, the cultures were diluted to an OD₆₀₀ nm of 0.2 and were grown at room temperature until the OD600 nm reached 0.8. IPTG was added to the cultures to a concentration of 0.4 mM and cultures were grown for an additional 3 h at room temperature. The cultures were then centrifuged at 10,000× g for 15 min, the supernatant was decanted and the resulting pellet was resuspended in 2 mL of breakage buffer (100 mM HEPES, pH 7.2; 10 % glycerol; 1 mM AEBSF; 1 μg/mL leupeptin; and 1 μg/mL pepstatin). Cells were broken by passage two times through a french press and the resulting extract was centrifuged at 16,000× g for 15 min at 4° C. The supernatant was carefully removed (soluble protein) and the pellet was resuspended in 1 mL of breakage buffer. An additional 1 mL of breakage buffer containing 12 mM CHAPS was added and after mixing and incubation on ice for 30 min, the mixture was centrifuged at 16,000× g for 15 min at 4° C. The supernatant was carefully removed (CHAPS soluble protein) and the pellet was resuspended in 2 mL of breakage buffer (insoluble protein).

Protein extracts (soluble, CHAPS soluble and insoluble) from TOP10 cells containing pTrcEtp, pTrcAds and pTrcHis-TOPO/lacZ were assayed by measuring the oxidation of NADPH or NADH using the decrease in absorbance at 340 nm (Hearl et al., J. Biol. Chem. 260:16361-16366 (1985)). Assays contained 100 mM potassium phosphate (pH 7.2), 10% glycerol, 1.5 mM succinic semialdehyde, 0.5 mM of either NADPH or NADH and from 10 μg-400 μg of enzyme extract in a total volume of 1 mL. Assays were initiated by addition of succinic semialdehyde and were measured at room temperature. Protein concentrations of each extract were determined using the BioRad protein assay (Biorad). Protein extracts were also analyzed by SDS-PAGE.

Enzyme assays showed that both GHBD1 (SEQ ID NO: 13) and GHBD2 (SEQ ID NO: 15) are most prevalent in the soluble protein fraction. Analysis of the commassie-stained protein gel confirmed these results. Some activity was found in the CHAPS soluble fraction but is most likely carry over from the soluble protein fraction and no activity was detected in the insoluble protein fraction. No activity was detected in any of the pTrcHis-TOPO/lacZ protein extracts. The oxidation of succinic semialdehyde is dependant on NADPH only, since no activity was found when NADH was used as electron donator. The specific activities for GHBD1 (SEQ ID NO: 13) and GHBD2 (SEQ ID NO: 15) were 0.14 μmol/min⁻¹/mg⁻¹ and 0.097 μmol/min⁻¹/mg⁻¹, respectively assuming an extinction coefficient for NADPH of 6.22×10³ M⁻¹ cm⁻¹ (Hearl et al., J. Biol. Chem. 260:16361-16366 (1985)).

Protein Purification and Kinetic Analysis:

Plasmids pTrcEtp, pTrcAds and pTrcHis2 containing gabT (described in Example 6) were retransformed into TOP10 cells and freshly plated transformants were inoculated into 2 mL of LB liquid media containing ampicillin (100 μg/mL). Cultures were grown at 37° C. for 5 h and then were transferred to 100 mL of LB liquid media containing ampicillin (100 μg/mL). These were grown overnight at room temperature. The next morning, 50 mL of each culture was transferred to 1 L of LB and grown at room temperature until the OD600 nm reached 0.8. IPTG was added to the cultures to a concentration of 0.4 mM and cultures were grown for an additional 4 h at room temperature. The cultures were then centrifuged at 10,000× g for 15 min, the supernatant was decanted and the resulting pellet was resuspended in 10 mL of breakage buffer (100 mM HEPES, pH 7.2; 10 % glycerol; 1 mM AEBSF; 1 μg/mL leupeptin; and 1 μg/mL pepstatin). Cells were broken by passage two times through a french press and the resulting extract was centrifuged at 16,000× g for 15 min at 4° C. The supernatant was carefully removed and 40 mL of Ni loading buffer (20 mM sodium phosphate, pH 7.5; 10 mM immidazole; 500 mM sodium chloride) was added. Proteins were loaded onto a 5 mL Hitrap chelating column (Pharmacia, Inc.) pre-equilibrated with 100 mM nickel sulfate and washed with 20 mL of loading buffer. Another wash with 50 mL of loading buffer containing 60 mM immidazole was carried out and bound proteins were eluted with a gradient of loading buffer containing immidazole from 60 mM to 1000 mM. All fractions were analyzed by SDS-PAGE.

All proteins eluted at around 120 mM immidazole. Purified proteins from pTrcAds and pTrcEtp were assayed with succinatesemialdehyde and NADPH as described above. The pH optimum for both proteins was found to be around pH 7.0. Assays contained 0.5 mM NADPH, 100 mM sodium phosphate (pH 7.0), 1 mg/mL BSA, succinatesemialdehyde and enzyme in 200 μL total. Assays were carried out at various concentrations of succinatesemialdehyde (0.1-30 mM) using 3 μg/mL of each enzyme. Under these conditions, the kinetic parameters for the Arabidopsis dehydrogenase (k_{cat}=1290 min-1, K_{M}=4 mM) and the Tulip dehydrogenase (k_{cat}=1200 min⁻¹, K_{M}=3 mM) were determined. The ablility of each enzyme to reduce to α-methylenesuccinatesemialdehyde to α-methylene-γ-hydroxybutyrate was assessed in a coupled assay. The GABT protein from E. coli, purified as described above, was used to generate α-methylenesuccinatesemialdehyde from α-methylene-γ-aminobutyrate. This assay was carried out in the presence or absence of the Arabidopsis and Tulip dehydrogenase. Reactions included 1 mM pyridoxal phosphate, 10 mM α-ketoglutarate, 100 mM potassium phosphate (pH 7.0), 0.5 mM NADPH, 1 mg/mL BSA, 100 μg purified GABT and 1 mM α-methylene-γ-aminobutyrate in 200 μL total volume. To this was added either the Arabidopsis or Tulip protein (20 μg/mL and 3 μg/mL) or no additional protein as negative control. Assays were incubated at room temperature for 30 min and the reaction was stopped with 10% HCl. Acidification of the assay also served to partially convert α-methylene-γ-hydroxybutyrate to α-methylene-γ-butyrolactone (MBL). MBL was separated on a Waters ion exclusion column (SH1011) with an isocratic gradient of 0.01 N H₂SO₄ over 30 min using a Waters 2690 HPLC. Compounds were detected with a Waters 9600 PDA detector. Both the Arabidopsis and Tulip dehydrogenases could convert α-methylenesuccinatesemialdehyde to \tilde{α}methylene-γ-aminobutyrate.

### Example 9

### Cloning and Characterization of Glucosyl Transferase

Two clones from the tulip pistil library, etp1c.pk001.n21 and etp1c.pk005.l14, and two EST sequences from the Alstroemeria emerging leaf library, eae1c.pk005.k7 and eae1c.pk006.e12 were identified that had homology to known UDP-glucosyltransferases. Plasmid DNA containing each cDNA was purified and the complete cDNA sequence was obtained for etp1c.pk001.n21, called n21 (SEQ ID NO: 17), etp1c.pk005.l14, called l14 (SEQ ID NO: 19), eae1c.pk005.k7, called k7 (SEQ ID NO: 21) and eae1c.pk006.e12, called e12 (SEQ ID NO: 23).

The DNA sequences were translated into their corresponding protein sequences (SEQ ID NO: 18), (SEQ ID NO: 20), (SEQ ID NO: 22) and (SEQ ID NO: 24), respectively, using EditSeq (DNASTAR, Inc.) and each was compared to sequences in the “nr” database using BLASTP analysis (Matrix, Blosum62; Gap existence cost, 11; Per residue gap cost, 1; Expect, 10; Descriptions, 50; Alignments, 50; Filter, None). Results of the comparison are shown in Table 8.

TABLE 8

| | | SEQ | SEQ | % | % | |
|---|---|---|---|---|---|---|
| EST | | ID | ID | Iden- | Simi- | E- |
| Name | Similarity Identified | Base | Peptide | tity^{a} | larity^{a} | value^{c} |
| N21 | gb\|AAF61647.1\|(AF190634) | 17 | 18 | 51 | 67 | e- |
| | UDP-glucose: salicylic acid | | | | | 136 |
| | glucosyltransferase | | | | | |
| | [Nicotiana tabacum] | | | | | |
| L14 | gb\|AAF61647.1\|(AF190634) | 19 | 20 | 51 | 69 | e- |
| | UDP-glucose: salicylic acid | | | | | 137 |
| | glucosyltransferase | | | | | |
| | [Nicotiana tabacum] | | | | | |
| K7 | gb\|AAF61647.1\|(AF190634) | 21 | 22 | 49 | 67 | e- |
| | UDP-glucose: salicylic acid | | | | | 128 |
| | glucosyltransferase | | | | | |
| | [Nicotiana tabacum] | | | | | |
| E12 | gb\|AAF61647.1\|(AF190634) | 23 | 24 | 50 | 68 | e- |
| | UDP-glucose: salicylic acid | | | | | 134 |
| | glucosyltransferase | | | | | |
| | [Nicotiana tabacum] | | | | | |

^{a}% Identity is defined as percentage of amino acids that are identical between two proteins.
^{b}% Similarity is defined as percentage of amino acids that are identical or conserved between two proteins.
^{c}Expect value. The Expect value estimates the statistical significance of the match, specifying the number of matches, with a given score, that are expected in a search of a database of this size absolutely by chance.

Cloning of Plant UDP-Glucosyltransferase ESTs into a Heterologous Expression System:

Primers N21-5 (SEQ ID NO: 59) and N21-3His (SEQ ID NO: 60) were used to amplify n21 (SEQ ID NO: 17). Primers L14-5Pag (SEQ ID NO: 61) and L14-3Xho (SEQ ID NO: 62) were used to amplify l14 (SEQ ID NO: 19). Primers K7-5Pag (SEQ ID NO: 63) and K7-3Hind (SEQ ID NO: 64) were used to amplify k7 (SEQ ID NO: 21). Primers E12-5Pag (SEQ ID NO: 65) and E12-3Hind (SEQ ID NO: 66) were used to amplify e12 (SEQ ID NO: 23). PCR products were gel purified (1% agarose gel in TAE buffer) using the QIAquick Gel Extraction Kit (Qiagen) and eluted with 50 μL of EB buffer (10 mM Tris-HCl, pH 8.0). PCR products were combined with 1 μL of pYES2.1/V5-His-TOPO vector (Invitrogen) and incubated at room temperature for 5 min. Two μL of the ligation mix was added to 50 μL of TOP10 chemically competent E. coli cells (Invitrogen). The cells were incubated on ice for 30 min, heated to 42° C. for 30 sec and incubated again on ice for 5 min. SOC media (250 μL) was added to each transformation and the mixture was incubated at 37° C. with shaking at 250 rpm for 1 hour. Subsequently, a series of 10 μL, 25 μL, 50 μL and 150 μL was plated onto LB media containing ampicillin (100 μg/mL). These plates were incubated overnight at 37° C. Colonies that grew on the selective media were randomly picked and transferred into 250 μL LB liquid media containing ampicillin (100 μg/mL); these colonies were grown for 3 hours at 37° C. with shaking at 250 rpm. Aliquots of these cultures were analyzed by PCR to screen for the insert in these colonies. Positive colonies were grown in 5 mL LB liquid media containing ampicillin (100 μg/mL) overnight at 37° C. with shaking at 250 rpm. Plasmid DNA was isolated using a miniprep kit (Qiagen). Plasmid DNA was subjected to restriction digest analysis to confirm the correct gene orientation in the plasmid. The plasmids that contained the correct gene were then transformed into Saccharomyces cerevisiae strain INVSc1 (Invitrogen); INVSc1 is auxotrophic for histidine, leucine, tryptophan and uracil. Transformants were plated on SC minimal media (lacking uracil and containing 2% glucose) and grown at 30° C. for 3 days. In addition, the pYES2.1/V5-His-TOPO/lacZ plasmid was also transformed into INVSc1 cells and was used as a negative control in expression and enzyme assay experiments. The UDP-glucosyltransferase ESTs and lacZ proteins were expressed with a C-terminal polyhistidine tag.

Expression Studies of UDP-Glucosyltransferases:

Freshly transformed S. cerevisiae colonies containing pYES2.1/V5-His-TOPO with UDP-glucosyltransferase inserts or pYES2.1/V5-His-TOPO/lacZ were inoculated into 50 mL of SC minimal media (lacking uracil and containing 2% glucose) and grown at 30° C. with shaking at 250 rpm. After about 16 hours of growth, the OD₆₀₀ was adjusted to 0.4 by the dilution of an aliquot into 50 mL of SC minimal media (lacking uracil and containing 2% galactose). The cultures were grown at 30° C. with shaking at 250 rpm for 5 hours. The cultures were then centrifuged at 5,000× g for 10 min and the supernatant was decanted; the resulting pellet was resuspended in 2 mL of buffer (100 mM Tris-HCl, 1 mM MgCl₂, 1 mM DTT, pH 7.5). Cells were broken by passage through a french press and the resulting extract was centrifuged at 13,000× g for 12 min at 4° C. The supernatant was removed (soluble protein) and the pellet (insoluble protein) was resuspended in 1 mL of SDS-PAGE loading buffer (50 mM Tris-HCl, 100 mM DTT, 2% SDS, 0.1% bromophenol blue, 10% glycerol, pH 6.8). Approximately 20 μg of total soluble protein and total insoluble protein was loaded onto a 4-12% Bis-Tris Gel (NuPAGE, Invitrogen). Proteins were transferred to a nitrocellulose membrane by Western blotting, and expression was confirmed by chemiluminescent detection of the polyhistidine fusion proteins using a India HisProbe (Pierce).

Soluble protein extracts were assayed for glucosyltransferase activity. The assay mixtures contained 100 mM potassium phosphate (pH 5.5), 20 mM Tulipalin (open-chain form), 20 mM UDP-glucose, and approximately 1,500 μg-2,000 μg of total soluble protein extract in a total volume of 500 μL. Assays were incubated at 37° C. for 90 min. The enzyme reactions were stopped by freezing to −20° C. Assays were subsequently thawed and (to remove protein) filtered using a 10,000 MWCO microcon filter (Amicon). The constituents of the assay were then analyzed using an LC/MS coupled system. Ten μL of the filtered assay was injected onto a C18 5μ Reverse Phase column (15 cm×2.1 mm) (Alltima, Alltech) on a Waters 2690 HPLC coupled to a Micromass Platform LCZ mass spectrometer with parameters optimized using a tuliposide standard purified from Alstroemeria. The following mobile phase method was utilized to separate the reaction components by HPLC: 0-22 min 100% H₂O, 22-27 min a linear gradient from 100% H₂O to 100% acetonitrile, 27-32 min 100% acetonitrile, and 32-42 min a linear gradient from 100% acetonitrile to 100% H₂O. In assays containing all components the tuliposide eluted at 12 min in a solvent of 100% H₂O. The tuliposide was detected as both the sodium ion (m/z 301) and the potassium ion (m/z 317) with the potassium ion being the dominant species. No activity (no tuliposide mass) was detected in any of the pYES2.1/V5-His-TOPO/lacZ protein extracts, confirming that the glucosyltransferase activity described was dependent on the expression of the plant glucosyltransferase ESTs.

What is claimed is:

1. An isolated nucleic acid fragment encoding a tuliposide A synthesizing protein selected from the group consisting of:
(a) an isolated nucleic acid fragment encoding the amino acid sequence set forth in SEQ ID NO: 2, SEQ ID NO: 4, SEQ ID NO: 6, SEQ ID NO: 8, SEQ ID NO: 10, SEQ ID NO: 14, SEQ ID NO: 18, SEQ ID NO: 20, SEQ ID NO: 22 and SEQ ID NO: 24;
(b) an isolated nucleic acid fragment that hybridizes with SEQ ID NO: 2, SEQ ID NO: 4, SEQ ID NO: 6, SEQ ID NO: 8, SEQ ID NO: 10, SEQ ID NO: 14, SEQ ID NO: 18, SEQ ID NO: 20, SEQ ID NO: 22 and SEQ ID NO: 24 Under the following hybridization conditions: 0.1× SSC, 0.1% SDS at 65° C., and washed with 2× SSC, 0.1% SDS followed by 0.1× SSC, 0.1% SDS; and
(c) an isolated nucleic acid fragment that is completely complementary to (a) or (b).

2. The isolated nucleic acid fragment of claim 1 selected from the group consisting of SEQ ID NO: 1, SEQ ID NO: 3, SEQ ID NO: 5, SEQ ID NO: 7, SEQ ID NO: 9, SEQ ID NO: 13, SEQ ID NO: 17, SEQ ID NO: 19, SEQ ID NO: 21, and SEQ ID NO: 23.

3. A polypeptide encoded by the isolated nucleic acid fragment of claim 1.

4. The polypeptide of claim 3 selected from the group consisting of SEQ ID NO: 2, SEQ ID NO: 4, SEQ ID NO: 6, SEQ ID NO: 8, SEQ ID NO: 10, SEQ ID NO: 14, SEQ ID NO: 18, SEQ ID NO: 20, SEQ ID NO: 22, and SEQ ID NO: 24.

5. A chimeric gene comprising the isolated nucleic acid fragment of claim 1 operably linked to suitable regulatory sequences.

6. The chimeric gene of claim 5 wherein the suitable regulatory sequence is selected from the group comprising CYC1, HIS3, GAL1, GAL10, ADH1, PGK, PHO5, GAPDH, ADC1, TRP1, URA3, LEU2, ENO, TPI, AOX1, lac, ara, tet, trp, IP_{L}, IP_{R}, T7, tac, trc, amy, apr, npr, nos, ocs, CaMV, the promoter of the small subunit (ss) of the ribulose-1,5-bisphosphate carboxylase from soybean, and the promoter of the chlorophyll a/b binding protein.

7. A vector comprising the chimeric gene of claim 5.

8. A transformed host cell comprising the chimeric gene of claim 5.

9. The transformed host cell of claim 8 wherein the host cell is selected from the group consisting of bacteria, yeast, filamentous fungi, algae, and green plants.

10. The transformed host cell of claim 9 wherein the host cell is selected from the group of genera consisting of Escherichia, Bacillus, Brevibacterium, Corynebacterium, Mycobacterium, Rhodococcus, Arthrobacter, Nocardia, Streptomyces, Actinomyces, Salmonella, Acinetobacter, Pseudomonas, Methylomonas, Methylobacter, Alcaligenes, Synechocystis, Anabaena, Thiobacillus, Methanobacterium, Klebsiella, Burkholderia, Sphingomonas, Comamonas, Aspergillus, Trichoderma, Saccharomyces, Pichia, Candida, and Hansenula.

11. The transformed host cell of claim 9 wherein the host cell is selected from the group consisting of Alstroemeria, tulip, soybean, rapeseed, sunflower, cotton, corn, tobacco, alfalfa, wheat, barley, oats, sorghum, rice, Arabidopsis, cruciferous vegetables, melons, carrots, celery, parsley, tomatoes, potatoes, strawberries, peanuts, grapes, grass seed crops, sugar beets, sugar cane, canola, millet, beans, peas, rye, flax, hardwood trees, softwood trees, and forage grasses.

12. An isolated nucleic acid fragment comprising a first nucleotide sequence encoding a polypeptide of at least 498 amino acids that has at least 78% identity based on the BLASTP method of alignment when compared to a polypeptide having the sequence as set forth in SEQ ID NO: 2 or a second nucleotide sequence comprising the complement of the first nucleotide sequence, wherein said enzyme has glutamate decarboxylase activity.

13. An isolated nucleic acid fragment comprising a first nucleotide sequence encoding a polypeptide of at least 509 amino acids that has at least 74% identity based on the BLASTP method of alignment when compared to a polypeptide having the sequence as set forth in SEQ ID NO: 4 or a second nucleotide sequence comprising the complement of the first nucleotide sequence, wherein said enzyme has glutamate decarboxylase activity.

14. An isolated nucleic acid fragment comprising a first nucleotide sequence encoding a polypeptide of at least 529 amino acids that has at least 74% identity based on the BLASTP method of alignment when compared to a polypeptide having the sequence as set forth in SEQ ID NO: 6 or a second nucleotide sequence comprising the complement of the first nucleotide sequence, wherein said enzyme has glutamate decarboxylase activity.

15. An isolated nucleic acid fragment comprising a first nucleotide sequence encoding a polypeptide of at least 471 amino acids that has at least 77% identity based on the BLASTP method of alignment when compared to a polypeptide having the sequence as set forth in SEQ ID NO: 8 or a second nucleotide sequence comprising the complement of the first nucleotide sequence, wherein said enzyme has □-aminobutyrate aminotransferase activity.

16. An isolated nucleic acid fragment comprising a first nucleotide sequence encoding a polypeptide of at least 507 amino acids that has at least 80% identity based on the BLASTP method of alignment when compared to a polypeptide having the sequence as set forth in SEQ ID NO: 10 or a second nucleotide sequence comprising the complement of the first nucleotide sequence, wherein said enzyme has □-aminobutyrate aminotransferase activity.

17. An isolated nucleic acid fragment comprising a first nucleotide sequence encoding a polypeptide of at least 290 amino acids that has at least 81% identity based on the BLASTP method of alignment when compared to a polypeptide having the sequence as set forth in SEQ ID NO: 14 or a second nucleotide sequence comprising the complement of the first nucleotide sequence, wherein said enzyme has γ-hydroxybutyrate dehydrognease activity.

18. An isolated nucleic acid fragment comprising a first nucleotide sequence encoding a polypeptide of at least 454 amino acids that has at least 51% identity based on the BLASTP method of alignment when compared to a polypeptide having the sequence as set forth in SEQ ID NO: 18 or a second nucleotide sequence comprising the complement of the first nucleotide sequence, wherein said enzyme has UDP-glucosyltransferase activity.

19. An isolated nucleic acid fragment comprising a first nucleotide sequence encoding a polypeptide of at least 454 amino acids that has at least 51% identity based on the BLASTP method of alignment when compared to a polypeptide having the sequence as set forth in SEQ ID NO: 20 or a second nucleotide sequence comprising the complement of the first nucleotide sequence, wherein said enzyme has UDP-glucosyltransferase activity.

20. An isolated nucleic acid fragment comprising a first nucleotide sequence encoding a polypeptide of at least 459 amino acids that has at least 49% identity based on the BLASTP method of alignment when compared to a polypeptide having the sequence as set forth in SEQ ID NO: 22 or a second nucleotide sequence comprising the complement of the first nucleotide sequence; wherein said enzyme has UDP-glucosyltransferase activity.

21. An isolated nucleic acid fragment comprising a first nucleotide sequence encoding a polypeptide of at least 459 amino acids that has at least 50% identity based on the BLASTP method of alignment when compared to a polypeptide having the sequence as set forth in SEQ ID NO: 24 or a second nucleotide sequence comprising the complement of the first nucleotide sequence, wherein said enzyme has UDP-glucosyltransferase activity.

22. A host cell comprising a partial or complete knockout of at least one tuliposide A synthesizing protein, the protein having an amino acid sequence selected from the group consisting of:
(a) SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24;
(b) an isolated nucleic acid molecule that hybridizes with SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, and SEQ ID NO: 24 under the following hybridization conditions: 0.1× SSC, 0.1% SDS, 65° C. and washed with 2× SSC, 0.1% SDS followed by 0.1× SSC, 0.1% SDS; or
(c) an isolated nucleic acid molecule that is complementary to (a) or (b).

23. A host cell of claim 22, wherein the host cell is a plant cell.

24. A compound α-methylenesuccinate seminaldehyde, represented by Formula I.

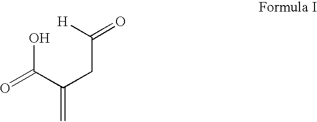

25. A method of producing a nucleic acid fragment encoding a tuliposide A synthesizing protein, the method comprising:
(a) probing a genomic library with the nucleic acid fragment selected from the group consisting of: the nucleic acid of claim 1, the nucleic acid as set forth in SEQ ID NO: 12 and the nucleic acid as set forth in SEQ ID NO: 16;
(b) identifying a cDNA clone that hybridizes with the nucleic acid fragment selected from the group consisting of: the nucleic acid of claim 1, the nucleic acid as set forth in SEQ ID NO: 12 and the nucleic acid as set forth in SEQ ID NO: 16; and
(c) sequencing the genomic fragment that comprises the clone identified in step (b), wherein the sequenced genomic fragment encodes a tuliposide A synthesizing protein.

26. A method of producing a nucleic acid fragment encoding a tuliposide A synthesizing protein, the method comprising:
(a) synthesizing at least one oligonucleotide primer corresponding to a portion of the sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 and SEQ ID NO: 23, and
(b) amplifying an insert present in a cloning vector using the oligonucleotide primer of step (a), wherein the amplified insert encodes a portion of an amino acid sequence encoding a tuliposide A synthesizing protein.

27. The product of the method of claims 25 or 26.

28. A method of producing α-methylene-γ-aminobutyrate comprising contacting a transformed host cell with an effective amount of γ-methyleneglutamate, said transformed host cell comprising a nucleic acid fragment encoding the polypeptide selected from the group consisting of:
(a) SEQ ID NO: 2, SEQ ID NO: 4, and SEQ ID NO: 6;
(b) an isolated nucleic acid molecule that hybridizes with SEQ ID NO: 2, SEQ ID NO: 4, and SEQ ID NO: 6 under the following hybridization conditions: 0.1× SSC, 0.1% SDS, 65° C. and washed with 2× SSC, 0.1% SDS followed by 0.1× SSC, 0.1% SDS; and
(c) an isolated nucleic acid molecule that is complementary to (a) or (b);
operably linked to suitable regulatory sequences.

29. A method of producing α-methylenesuccinate seminaldehyde comprising contacting a transformed host cell with an effective amount of α-methylene-γ-aminobutyrate, said transformed host cell comprising a nucleic acid fragment encoding the polypeptide selected from the group consisting of:
(a) SEQ ID NO: 8, SEQ ID NO: 10, and SEQ ID NO: 12, operably linked to suitable regulatory sequences;
(b) an isolated nucleic acid molecule that hybridizes with SEQ ID NO: 8, SEQ ID NO: 10, and SEQ ID NO: 12 under the following hybridization conditions: 0.1× SSC, 0.1% SDS, 65° C. and washed with 2× SSC, 0.1% SDS followed by 0.1× SSC, 0.1% SDS; and
(c) an isolated nucleic acid molecule that is complementary to (a) or (b);
operably linked to suitable regulatory sequences.

30. A method of producing α-methylene-γ-hydroxybutyrate comprising contacting a transformed host cell with an effective amount α-methylenesuccinate seminaldehyde, said transformed host cell comprising a nucleic acid fragment encoding the polypeptide selected from the group consisting of:
(a) SEQ ID NO: 14 and SEQ ID NO: 16;
(b) an isolated nucleic acid molecule that hybridizes with SEQ ID NO: 14 and SEQ ID NO: 16 under the following hybridization conditions: 0.1× SSC, 0.1% SDS, 65° C. and washed with 2× SSC, 0.1% SDS followed by 0.1× SSC, 0.1% SDS; and
(c) an isolated nucleic acid molecule that is complementary to (a) or (b);
operably linked to suitable regulatory sequences.

31. A method of producing tuliposide A comprising contacting a transformed host cell with an effective amount of α-methylene-γ-hydroxybutyrate, said transformed host cell comprising a nucleic acid fragment encoding the polypeptide selected from the group consisting of:
(a) SEQ ID NO: 18, SEQ ID NO: 20, SEQ ID NO: 22 and SEQ ID NO: 24;
(b) an isolated nucleic acid molecule that hybridizes with SEQ ID NO: 18, SEQ ID NO: 20, SEQ ID NO: 22, and SEQ ID NO: 24 under the following hybridization conditions: 0.1× SSC, 0.1% SDS, 65° C. and washed with 2× SSC, 0.1% SDS followed by 0.1× SSC, 0.1% SDS; and
(c) an isolated nucleic acid molecule that is complementary to (a) or (b);
operably linked to suitable regulatory sequences.

32. A method for producing tuliposide A and tuliposide A pathway intermediates, the method comprising contacting a transformed host cell under suitable growth conditions with an effective amount of γ-methyleneglutamate, said transformed host cell comprising a tuliposide A synthesizing protein selected from the group consisting of:
(a) SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22,
SEQ ID NO: 24 and mixtures thereof;
(b) an isolated nucleic acid molecule that hybridizes with SEQ ID NO: 2, SEQ ID NO: 4, and SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 under the following hybridization conditions: 0.1× SSC, 0.1% SDS, 65° C. and washed with 2× SSC, 0.1% SDS followed by 0.1× SSC, 0.1% SDS; and
(c) an isolated nucleic acid molecule that is complementary to (a) or (b);
operably linked to suitable regulatory sequences.

33. The method of claim 32 wherein the tuliposide A pathway intermediates are selected from the group consisting of α-methylene-γ-aminobutyrate, α-methylenesuccinate semialdehyde, and α-methylene-γ-hydroxybutyrate.

34. A method of altering the level of expression of a tuliposide A synthesizing protein in a host cell comprising:
(a) transforming a host cell with a chimeric gene selected from the group consisting of:
(i) the chimeric gene of claim 5;
(ii) a chimeric gene comprising the isolated nucleic acid fragment encoding the amino acid sequence set forth in SEQ ID NO: 12, operably linked to suitable regulatory sequences; and
(ii) a chimeric gene comprising the isolated nucleic acid fragment encoding the amino acid sequence set forth in SEQ ID NO: 16, operably linked to suitable regulatory sequences; and
(b) growing the transformed host cell produced in step (a) under conditions that are suitable for expression of the chimeric gene.

35. The method of claim 34 wherein the level of expression of the tuliposide A synthesizing protein is enhanced.

36. The method of claim 35 wherein the level of expression is enhanced by expression on a multicopy plasmid or association with suitable regulatory sequences.

37. The method of claim 34 wherein the level of expression of the tuliposide A synthesizing protein is decreased.

38. The method of claim 37 wherein the tuliposide A synthesizing protein is expressed in antisense orientation.

39. The method of claim 37 wherein the level of expression of tuliposide A synthesizing protein is decreased by disruption by insertion of foreign DNA into the coding region or disruption by point mutation or deletion.

40. A method of producing a mutated microbial gene sequence encoding a protein having an altered biological activity, the method comprising the steps of:
(a) digesting a mixture of nucleotide sequences with restriction endonucleases wherein said mixture comprises:
(i) a native microbial gene selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, and SEQ ID NO: 23;
(ii) a first population of nucleotide fragments which hybridizes to the native microbial gene sequence;
(iii) a second population of nucleotide fragments which does not hybridize to said native microbial sequence;
wherein a mixture of restriction fragments are produced; (b) denaturing the mixture of restriction fragments obtained in step (a); (c) incubating the denatured mixture of restriction fragments of step (b) with a polymerase; and (d) repeating steps (b) and (c) wherein a mutated microbial gene sequence is produced encoding a protein having an altered biological activity.

41. A mutated microbial gene sequence encoding a protein having an altered biological activity produced by the method of claim 44.

42. A method for producing tuliposide A, in an aqueous reaction mixture, comprising the steps of:
(a) contacting γ-methyleneglutamate with γ-methyleneglutamate decarboxylase;
(b) contacting the product of step (a) with \tilde{α}-methylene-γ-aminobutyrate aminotransferase;
(c) contacting the product of step (b) with α-methylene-γ-hydroxybutyrate dehydrogenase;
(d) contacting the product of step (c) with α-methylene-γ-hydroxybutyrate/UDP-glucose glucosyltransferase to produce tuliposide A; and
(e) isolating the tuliposide A produced in step (d).

43. The method of claim 42 wherein the product of step (a) comprises α-methylene-γ-aminobutyrate; the product of step (b) comprises α-methylenesuccinate semialdehyde; and the product of step (c) comprises α-methylene-γ-hydroxybutyrate.

44. A method of producing α-methylene-γ-aminobutyrate comprising contacting γ-methyleneglutamate with γ-methyleneglutamate decarboxylase under suitable enzymatic conditions in an aqueous reaction mixture and isolating the α-methylene-γ-aminobutyrate produced.

45. A method of producing α-methylenesuccinate semialdehyde comprising contacting α-methylene-γ-aminobutyrate with \tilde{α}methylene-γ-aminobutyrate aminotransferase under suitable enzymatic conditions in an aqueous reaction mixture and isolating the α-methylenesuccinate semialdehyde produced.

46. A method of producing \tilde{α}methylene-γ-hydroxybutyrate comprising contacting \tilde{α}methylenesuccinate semialdehyde with α-methylene-γ-hydroxybutyrate dehydrogenase under suitable enzymatic conditions in a suitable aqueous reaction mixture and isolating the α-methylene-\tilde{γ}hydroxybutyrate produced.

47. A method of producing tuliposide A comprising contacting α-methylene-γ-hydroxybutyrate with α-methylene-γ-hydroxybutyrate/UDP-glucose glucosyltransferase under suitable enzymatic conditions in an aqueous reaction mixture and isolating the tuliposide A produced.

48. A method of producing tulipalin A comprising contacting α-methylene-γ-hydroxybutyrate with a catalytic amount of a strong acid catalyst and isolating the tulipalin A produced.

49. The method of claim 44, 45, 46 or 47 wherein the enzyme catalyst is in the form of whole microbial cells, permeabilized microbial cells, one or more cell components of a microbial cell extract, partially purified enzyme(s), or purified enzyme(s).

50. The method of claim 44, 45, 46 or 47 wherein the enzyme catalyst is immobilized in a polymer matrix or on a soluble or insoluble catalyst support.
